(19) United States
(12) Patent Application Publication
(10) Pub. No.: US 2009/0304827 A1
(43) Pub. Date: Dec. 10, 2009

(54) Combinations of Ingredients Having Synergistic Anti-Inflammatory Effects

(76) Inventor: Darrick S.H.L. Kim, Schaumburg, IL (US)

Correspondence Address: MARSHALL, GERSTEIN & BORUN LLP, 233 SOUTH WACKER DRIVE, 6300 SEARS TOWER, CHICAGO, IL, 60606-6357, US

(21) Appl. No.: 12/463,043

(22) Filed: May 8, 2009

Related U.S. Application Data
(60) Provisional application No. 61/051,640, filed on May 8, 2008.
(60) Provisional application No. 61/056,205, filed on May 27, 2008.

(51) Int. Cl. A61K 36/00; A61K 31/12; A61K 31/202

(52) U.S. Cl. 424/725; 514/679; 514/560

(57) ABSTRACT

Nutritional supplement compositions comprising selected herbs and/or herbal constituents thereof that provide synergistic anti-inflammatory effects are disclosed.

### CROSS-REFERENCE TO RELATED APPLICATION

The present application claims the benefit of priority of U.S. Provisional Application No. 61/051,640, filed May 8, 2008 and U.S. Provisional Application No. 61/056,205, filed May 27, 2008, the disclosure of which is incorporated herein by reference in its entirety

### FIELD OF THE INVENTION

The present invention relates to nutritional supplement composition designed to provide synergistic anti-inflammatory effects and methods of their use. More particularly, the present invention relates to nutritional supplement useful for alleviating pain induced by inflammation. The present invention also relates generally to processed herbal composition and/or herbal constituents thereof exhibiting synergistic inhibition of the expression and/or activity of inducible cyclooxygenase-2 (COX-2) and/or LPS-induced NO production and/or iNOS enzyme activity and/or LPS-induced PGE2 production.

### BACKGROUND OF THE INVENTION

### Inflammation

More than fifty million Americans suffer pain induced by inflammatory diseases. In modern non-herbal medicine, there are two major categories of anti-inflammatory medicines: steroidal and non-steroidal. Steroidal anti-inflammatory medicines are based on hormonal substances, such as cortisone. Steroidal medications have a stronger anti-inflammatory response than non-steroidal medicines.

Non-steroidal anti-inflammatory drugs have three major actions, all of which are related to inhibition of cyclo-oxygenase resulting in decreased formation of prostanoids. Firstly, an anti-inflammatory action is achieved by reducing production of vasodilator prostaglandins (PGE2, PGI2), thus reducing inflammation through oedema. Secondly, an analgesic effect is achieved by reduced prostaglandin production (less sensitization of nociceptic nerve endings to the inflammatory mediators brakykinin and 5-hydroxytryptamine). Thirdly, an antipyretic effect can produce an anti-inflammatory action, due to a decrease in the mediator PGE2 generated in response to inflammatory pyrogens, much as interleukin-1. There are many non-steroidal anti-inflammatory medications. Acetaminophen, aspirin, ibuprofen, and naproxen are the most commonly used non-steroidal anti-inflammatory medications.

There are side effects to both of these groups of medicines that include stomach upset, stomach bleeding, or ulcers, kidney problems, hearing problems and ankle swelling. Additionally, the steroidal anti-inflammatory medications have shown serious side effects including: loss of bone mass, cataracts, reduced ability to fight infection, swelling and weight gain, mood changes, high blood pressure, and problems with the bone marrow where blood cells are produced.

### Cyclooxygenase and Inflammation

Cyclooxygenase (COX) is an enzyme that is responsible for formation of biological mediators called prostanoids. Pharmacological inhibition of COX can provide relief from the symptoms of inflammation and pain.

COX converts arachidonic acid (AA, an w-6 PUFA) to prostaglandin H₂ (PGH₂), the precursor of the series-2 prostanoids. The enzyme contains two active sites: a heme with peroxidase activity, responsible for the reduction of PGG₂ to PGH₂, and a cyclooxygenase site, where arachidonic acid is converted into the hydroperoxy endoperoxide prostaglandin G₂ (PGG₂).

Currently three COX isoenzymes are known—COX-1, COX-2 and COX-3. COX-3 is a splice variant of COX-1 which retains intron one and has a frameshift mutation, thus some prefer the name COX-1b or COX-1 variant (COX-1v).

Different tissues express varying levels of COX-1 and COX-2. Although both enzymes act basically in the same fashion, selective inhibition can make a difference in terms of side-effects. COX-1 is considered a constitutive enzyme, being found in most mammalian cells. More recently it has been shown to be upregulated in various carcinomas and to have a central role in tumorigenesis. COX-2, on the other hand, is undetectable in most normal tissues. It is an inducible enzyme, becoming abundant in activated macrophages and other cells at sites of inflammation.

COX-2 gene expression is under the control of pro-inflammatory cytokines and growth factors. Thus, the inference is that COX-2 functions in both inflammation and control of cell growth. While COX-2 is inducible in many tissues, it is present constitutively in the brain and spinal cord, where it may function in nerve transmission for pain and fever. The two isoforms of COX are nearly identical in structure but have important differences in substrate and inhibitor selectivity and in their intracellular locations. Protective PGs, which preserve the integrity of the stomach lining and maintain normal renal function in a compromised kidney, are synthesized by COX-1. On the other hand, PGs synthesized by COX-2 in immune cells are central to the inflammatory process.

Both COX-1 and -2 oxygenate two other essential fatty acids—DGLA (ω-6) and EPA (ω-3)- to give the series-1 and series-3 prostanoids, which are less inflammatory than those of series-2. DGLA and EPA are competitive inhibitors with AA for the COX pathways. This inhibition is a major mode of action in the way that dietary sources of DGLA and EPA (e.g. borage, fish oil) reduce inflammation.

The main COX inhibitors are the non-steroidal anti-inflammatory drugs (NSAIDs). NSAIDs inhibit COX activity by excluding access for AA into the channel. The classical COX inhibitors are non-selective (i.e. they inhibit all types of COX), and the main adverse effects of their use are peptic ulceration and dyspepsia. It is believed that this may be due to the “dual-insult” of NSAIDs—direct irritation of the gastric mucosa (many NSAIDs are acids), and inhibition of prostaglandin synthesis by COX-1. Prostaglandins have a protective role in the gastrointestinal tract, preventing acid-insult to the mucosa.

Pro-inflammatory cytokines and mitogens, such as interleukin (IL)-1beta (IL-1b), interferon gamma, and tumour necrosis factor-alpha (TNF-a) induce COX-2. in addition, COX-2 expression in macrophages can also be stimulated by platlet activating factor (PAF) and PGE2.

A key role for COX-2 in joint inflammation is suggested through the up-regulation of COX-2 expression by cytokines in monocytes, macrophages, chondrocytes, osteoblasts, and synovial microvessel endothelial cells. On the other hand, reduced expression of COX-2 occurs after exposure to the anti-inflammatory cytokines IL-4, IL-10 and IL-13, as well as dexamethasone.

As COX-2 is induced by inflammatory stimuli and cytokines, the anti-inflammatory actions of NSAIDs are results of the inhibition of COX-2, while the unwanted side effects, such as gastric and intestinal mucosal damage and renal toxicity, are results of the inhibition of COX-1.

Diseases Associated with Inflammation and Pain

Millions of people and animals suffer pain due to inflammation of connective tissue, including joints and other body parts. The suffering that is encountered ranges from mild soreness to debilitating pain that prevents any motion of the afflicted body part. Connective tissue is present in all organs, so the term connective tissue diseases describes a group of diseases which influence a wide range of different body systems.

The term arthritis also refers to conditions where inflammation occurs in a joint. While some in the field use the term “arthritis” to strictly mean inflammation of the joints, the term is used more generally for almost all joint problems. So the term “inflammatory arthritis” generally means those diseases of joints where, for example, the immune system is causing inflammation in the joint. The characteristic symptoms of inflammatory arthritis are pain and swelling of one or more joints. Among the more common types of inflammatory arthritis are rheumatoid arthritis, gout, psoriatic arthritis (associated with the skin condition psoriasis), reactive arthritis, viral or post-viral arthritis (occurring after an infection), and spondylarthritis that affects the spine as well as joints. Arthritis is further subdivided into inflammatory and non-inflammatory arthritis.

One of the most common forms of connect tissue disease is osteoarthritis which is a non-inflammatory type of arthritis. Osteoarthritis is common in older age groups and is generally considered to be due to “wear and tear” of the joints leading to damage of the joint surfaces that results in pain on movement of the joint. There are many factors influencing development of osteoarthritis, including a family history of osteoarthritis and previous damage to the joint caused by injury or surgery.

Symptoms in osteoarthritis tend to get worse with activity, so that the greatest pain is experienced at the end of the day. In contrast, the symptoms of inflammatory arthritis include the greatest pain occurring at the movement of a joint after a night's sleep of inactivity. In the case of osteoarthritis, there may be hard, bony swelling of the joints, and a gritty feeling (or even noise) when the joint is moved.

Rheumatic diseases, one type of connective tissue diseases, that involve joints and the surrounding tissues such as ligaments, tendons and muscles, in addition to eyes, skin and glands. Rheumatic diseases are usually divided into those that primarily involve joints, known as arthritis, and those of other connective tissue diseases.

There are many common minor pains that are not arthritis but are due to injury, strain or inflammation of tendons or ligaments and are referred to as “soft tissue rheumatism.” Some of the more common soft tissue rheumatism conditions include tennis elbow, frozen shoulder, carpal tunnel syndrome, plantar fasciitis, and Achilles tendonitis.

Tennis elbow is due to inflammation of the tendons of the hand gripping muscles where these tendons are attached to the elbow. This results in pain at the elbow, worse on gripping with the hand, and the afflicted area is tender when pressed. It usually gets better by itself if the hand is rested, but the condition can recur. As is well appreciated, tennis elbow is not confined to tennis players.

Frozen shoulder is a stiffening of the ligaments around the shoulder joint that usually comes on after prolonged unaccustomed use of the arm. When afflicted with frozen shoulder, it is painful and difficult to move an arm in any direction. Past treatment has included a program of exercises to slowly increase the range of movement of the arm with a steroid injection into the shoulder to get it moving again.

Carpal tunnel syndrome involves a nerve that passes through the carpal tunnel on the front of the wrist into the hand. When this tunnel becomes inflamed it can press on the nerve causing shooting pain into the thumb and first two fingers. The syndrome can arise due to many conditions such as thyroid disease, pregnancy and arthritis.

Plantar fasciitis involves ligaments in the sole of the foot whose inflammation leads to pain on the bottom of the heel on walking.

Achilles tendonitis involves the Achilles tendon located at the back of the ankle that becomes inflamed and painful when walking or especially painful to stand up on tip-toe. This condition is usually caused by shoes that rub at the back of the heel.

### Herbs and Active Constituents Thereof

### Atractylodes Macrocephala or A. Ovata

Atractylodes is the dried or steam-dried rhizome (rootstalk) of Atractylodes macrocephala or A. ovata, perennial north Asian herbs in the Compositae family that grows in mountain valleys, especially in China's Zhejiang province. Atractylodes is thought to dry dampness, strengthen the Spleen or digestion, and promote diuresis, the formation and excretion of urine. It is used for diarrhea, generalized aching, mental fatigue, dizziness, lack of appetite, vomiting, edema (accumulation of fluids), and spontaneous sweating. It is also used to prevent miscarriage and to treat restless fetal movement. Other uses include restoring deficient digestion associated with poor absorption, malnutrition, anorexia, metabolic acidosis, hypoglycemia, and rheumatism.

### Atragalus Membranaceus, Fabaceae

Astragalus membranaceus, also known as huang qi is an herb of the family Fabaceae, is believed to act as a tonic to protect the immune system, improve the functioning of the lungs, adrenal glands and the gastrointestinal tract. In addition, it is also reputed to increase metabolism, sweating, promote healing and to reduce fatigue.

### Camellia Sinensis

Green tea is the dried leaves and leaf buds of the shrub Camellia sinensis. It is mainly produced in China and Japan. Dried tea leaves are composed mainly of phytochemicals known as polyphenols (36%), principally flavonols (including catechins), flavonoids, and flavondiols. The leaves also contain plant alkaloids (about 4%), including caffeine, theobromine and theophylline. Much of the research on green tea has been focused on its potential to prevent cancer. Research suggests that the polyphenols in green tea are responsible for a chemopreventive effect.

Cinnamomum Verum, Synonym C. zeylanicum

Cinnamon (Cinnamomum verum, synonym C. zeylanicum) is a small evergreen tree belonging to the family Lauraceae, exclusively native to Sri Lanka and very small parts of South India. Cinnamon has been suggested to improve blood glucose and cholesterol levels in people with Type 2 diabetes. The cinnaldehyde in cinnamon has been noted to prevent unwanted clumping of blood platelets by inhibiting the release of called arachidonic acid, an inflammatory fatty acid, from platelet membranes and reducing the formation of called thromboxane A2, an inflammatory messaging molecule. Cinnamon's ability to lower the release of arachidonic acid from cell membranes puts it in the category of an “anti-inflammatory” food. Cinnamon has also been noted for anti-microbial activity.

Curcuma longa, Zingiberaceae

Turmeric (Curcuma longa) is a rhizomatous herbaceous perennial plant of the ginger family, Zingiberaceae which is native to tropical South Asia. It is a significant ingredient in most commercial curry powders. Turmeric is widely used as a spice in Indian and other South Asian cooking.

In the Ayurvedic medicine, turmeric is thought to have many medicinal properties and many in India use it as a readily available antiseptic for cuts and burns. It is also used as an antibacterial agent. It is taken in some Asian countries as a dietary supplement, which allegedly helps with stomach problems and other ailments. It is only in recent years that Western scientists have increasingly recognized the medicinal properties of turmeric. It is currently being investigated for possible benefits in Alzheimer's disease, cancer and liver disorders.

A yellow pigmented fraction isolated from the rhizomes of C. longa contains curcuminoids. They are considered the most important active ingredients and are believed to be responsible for the biological activity of C. longa. The anti-inflammatory activity of curcuminoids has been evaluated in inflammatory reaction models such as chemical or physical irritants like carrageenin, cotton pellets, formaldehyde and the granuloma pouch. Human, double-blinded, clinical trials have demonstrated efficacy in rheumatoid arthritis at a dose of 1200 mg curcuminoids/day for five to six weeks.

Curcumin is thought to be a powerful antinociceptive (pain-relieving) agent. The effectiveness of turmeric in the reduction of joint inflammation, and recommended clinical trials as a possible treatment for the alleviation of arthritis symptoms. It is thought to work as a natural inhibitor of the cox-2 enzyme, and has been shown effective in animal models for neuropathic pain secondary to diabetes, among others. Though their major activity is anti-inflammatory, curcuminoids have been reported to possess antioxidant, antiallergic, wound healing, antispasmodic, antibacterial, antifungal and antitumor activity as well. Other curcuminoids isolated from C. longa include demethoxycurcumin, bisdemethoxycurcumin, a cis-trans geometrical isomer of curcumin. Curcuminoids may be found in other botanicals in addition to C. longa, such as C. aromatica, C. xanthorrhiza, C. zedoaria and etc.

Cuscuta chinensis, Convolvulaceae

Cuscuta chinensis (The ripe seed of Cuscuta chinensis Lam., an annual voluble parasitic herb of the family Convolvulaceae) is a perennial plant that ranges E. Asia, China, Japan and Korea. It is a parasitic vine that wraps around other plants for nourishment. Cuscuta seed is used in China for kidney deficiency. A lotion made from the stems is used in the treatment of sore heads and inflamed eyes. The seed is aphrodisiac, demulcent, diaphoretic, hepatic and tonic. It is decocted and used with other herbs to treat a variety of ailments. In particular, it is used in the treatment of impotence, nocturnal emissions, vertigo, lumbago, leucorrhoea, frequent micturation, decreased eyesight, threatened abortion and chronic diarrhea.

### Flax Seed

Flax (also known as Common Flax or Linseed) is a member of the genus Linum in the family Linaceae. Flax is native to the region extending from the eastern Mediterranean to India and was probably first domesticated in the Fertile Crescent. It was extensively cultivated in ancient Egypt.

Flax is grown both for its seeds and for its fibers. Various parts of the plant have been used to make fabric, dye, paper, medicines, fishing nets and soap. It is also grown as an ornamental plant in gardens.

Flax seeds come in two basic varieties, brown and yellow or golden, with most types having similar nutritional values and equal amounts of short-chain omega-3 fatty acids. The exception is a type of yellow flax called Linola or solin, which has a completely different oil profile and is very low in omega-3. Flax seeds produce a vegetable oil known as flaxseed or linseed oil; it is one of the oldest commercial oils and solvent-processed flax seed oil has been used for centuries as a drying oil in painting and varnishing.

Flax seeds contain high levels of lignans and Omega-3 fatty acids. Lignans may benefit the heart, possess anti-cancer properties and studies performed on mice found reduced growth in specific types of tumours. Initial studies suggest that flaxseed taken in the diet may benefit individuals with certain types of breast cancer, and also patients with prostate cancer. Flax may also lessen the severity of diabetes by stabilizing blood-sugar levels.

Harpagophytum procumbens, Pedaliaceae

Harpagophytum procumbens, Pedaliaceae also called grapple plant, wood spider and most commonly Devil's Claw, is a plant of the sesame family, native to South Africa that got its name from the peculiar appearance of its hooked fruit. The plant's large tuberous roots are used medicinally to reduce pain and fever, and to stimulate digestion. European colonists brought Devil's Claw home where it was used to treat arthritis.

The two active ingredients in Devil's Claw are Harpagoside and Beta sitosterol that possess anti-inflammatory properties and used for chronic treatment of pain. Devil's Claw is thought to have analgesic, sedative and diuretic properties. Devil's Claw is also claimed to be beneficial for treating diseases of the liver, kidneys, gallbladder and bladder, arthritis and rheumatism. It is said to help alleviate problems with and improve the vitality of the joints, as well as stimulating appetite and aid digestion, increase cholesterol and fatty acids in the blood.

A series of small-scale studies completed in Germany found that H. procumbens was indistinguishable from Vioxx in the treatment of chronic low back pain, and was well-tolerated after more than four years of treatment of H. procumbens alone. H. procumbens also seems efficacious in the treatment of arthritis-caused hip and knee pain.

Lycium chinense, Solanaceae

Wolfberry is the common name for the fruit of two very closely related species: Lycium barbarum and L. chinense, two species of boxthorn in the family Solanaceae (which also includes the potato, tomato, eggplant, deadly nightshade, chili pepper, and tobacco). Although its original habitat is obscure (probably southeastern Europe to southwest Asia), wolfberry species are now grown around the world, primarily in China.

Wolfberries have long played important roles in traditional Chinese medicine (TCM) where they are believed to enhance immune system function, improve eyesight, protect the liver, boost sperm production and improve circulation, among other effects.

### Omega-3 Fatty Acid

ω-3 fatty acids (commonly spelled omega-3 fatty acids) are a family of polyunsaturated fatty acids which have in common a carbon-carbon double bond in the ω-3 position.

Important nutritionally essential ω-3 fatty acids are: α-linolenic acid (ALA), eicosapentaenoic acid (EPA), and docosahexaenoic acid (DHA). The human body cannot synthesize ω-3 fatty acids de novo, but it can form 20- and 22-carbon unsaturated ω-3 fatty acids from the eighteen-carbon ω-3 fatty acid, α-linolenic acid. These conversions occur competitively with ω-6 fatty acids, which are essential closely related chemical analogues that are derived from linoleic acid. Both the ω-3α-linolenic acid and ω-6 linoleic acid are essential nutrients which must be obtained from food.

The U.S. Food and Drug Administration gave “qualified health claim” status to eicosapentaenoic acid (EPA) and docosahexaenoic acid (DHA) ω-3 fatty acids, stating that “supportive but not conclusive research shows that consumption of EPA and DHA ω-3 fatty acids may reduce the risk of coronary heart disease.

Some benefits have been reported in such conditions as rheumatoid arthritis and cardiac arrhythmias.

### Omega-6 Fatty Acid

ω-6 fatty acids (also spelled n-6 or omega-6 fatty acids) are a family of polyunsaturated fatty acids which have in common a carbon-carbon double bond in the Ω-6 position; that is, the sixth carbon from the end of the fatty acid.

The biological effects of the ω-6 fatty acids are largely mediated by their interactions with the ω-3 fatty acids.

Linoleic acid (18:2), the shortest chain omega-6 fatty acid, is an essential fatty acid. Arachidonic acid (20:4) is a physiologically significant ω-6 fatty acid and is the precursor for prostaglandins and other physiologically active molecules.

Some medical research has suggested that excessive levels of ω-6 fatty acids, relative to ω-3 fatty acids, may increase the probability of a number of diseases and depression. Modern Western diets typically have ratios of ω-6 to ω-3 in excess of 10 to 1, some as high as 30 to 1. The optimal ratio is thought to be 4 to 1 or lower.

### Dietary Sources of ω-6 Fatty Acids Include:

nuts
cereals
whole-grain breads
most vegetable oils
eggs and poultry
baked goods

List of ω-6 fatty acids

| Common name | Lipid name | Chemical name |
|---|---|---|
| Linoleic acid | 18:2 (n-6) | 9,12-octadecadienoic acid |
| Gamma-linolenic | 18:3 (n-6) | 6,9,12-octadecatrienoic acid |
| acid | | |
| Eicosadienoic acid | 20:2 (n-6) | 11,14-eicosadienoic acid |
| Dihomo-gamma- | 20:3 (n-6) | 8,11,14-eicosatrienoic acid |
| linolenic acid | | |
| Arachidonic acid | 20:4 (n-6) | 5,8,11,14-eicosatetraenoic acid |
| Docosadienoic acid | 22:2 (n-6) | 13,16-docosadienoic acid |
| Adrenic acid | 22:4 (n-6) | 7,10,13,16-docosatetraenoic acid |
| Docosapentaenoic | 22:5 (n-6) | 4,7,10,13,16-docosapentaenoic acid |
| acid | | |
| Calendic acid | 18:3 (n-6) | 8E,10E,12Z-octadecatrienoic acid |

### Resveratrol

Resveratrol, naringenin and naringin are naturally occurring flavonoids in grapes and grapefruits. these flavonoids on the induction of NO synthase (NOS) in RAW 264.7 cells activated with bacterial lipopolysaccharide (LPS, 50 ng ml⁻¹) were investigated. Resveratrol was found strongly to inhibit NO generation in activated macrophages, as measured by the amount of nitrite released into the culture medium, and resveratrol strongly reduced the amount of cytosolic iNOS protein and steady state mRNA levels.

### Ribes Nigrum

The Blackcurrant (Ribes nigrum) is a species of Ribes berry native to central and northern Europe and northern Asia. In French it is called a “cassis”.

In addition to the high levels of vitamin C, studies have also shown concentrated blackcurrant to be an effective Monoamine oxidase inhibitor (MAOI). Blackcurrant seed oil is a rich source of gamma-linolenic acid (GLA), a rare essential fatty acid.

### Rosmarinus Officinalis, Lamiaceae

Rosemary (Rosmarinus officinalis), native to the Mediterranean region, is a woody, perennial herb with fragrant evergreen needle-like leaves. It is a member of the mint family Lamiaceae. Forms range from upright to trailing; the upright forms can reach 1.5 m tall, rarely 2 m. The fresh and dried leaves are used frequently in traditional Mediterranean cuisine as an herb; they have a bitter, astringent taste, which compliments a wide variety of foods. Rosemary has been used to treat inflammation and was found to support iNOS inhibition, thus inhibiting the body's over-production of nitric oxide and the COX-2 enzyme.

### Salvia Officinalis, Lamiaceae

Sage (Salvia officinalis) is a genus of plants in the mint family, Lamiaceae. This genus includes approximately 700 to 900 species of shrubs, herbaceous perennials, and annuals with almost world-wide distribution. Sage has been known to strengthen the lungs and can be used in teas or tinctures to prevent coughs. Species of Salvia has also been used as anti-inflammatories to provide relief. Salvia officinalis tincture showed anti-inflammatory effects by reducing marrow acute phase response and NO synthesis.

### Scutellaria Baicalensis

Medicinal plants have been used as traditional remedies for hundreds of years. Scutellaria baicalensis Georgi (Huang Qui) is one of the important medicinal herbs widely used for the treatment of various inflammatory diseases, hepatitis, tumors, and diarrhea in East Asian countries such as China, Korea, Taiwan, and Japan.

Oroxylin A (a polyphenolic compound) isolated from Huang Qui was found to be a potent inhibitor of LPS-induced NO and PGE₂ productions by blocking iNOS and COX-2 gene activation. Baicalin, baicalein, and wogonin significantly inhibited LPS-induced NO production and iNOS gene expression in a concentration-dependent manner, but did not inhibit iNOS enzyme activity. Furthermore, wogonin, but not baicalin or baicalein, inhibited LPS-induced PGE₂ production and COX-2 gene expression.

### Tanacetum Parthenium and T. Vulgare

Feverfew, Tanacetum parthenium (L.) Schultz Bip. (Williams, Hoult, Harbome, Greenham, & Eagles, 1995). The anti-inflammatory activity of the major flavonoid, called tanetin, was found to be significant, particularly because feverfew is currently used in the treatment of arthritis and migraine. Tanacetum vulgare L. is a well-known medicinal plant with report on their anti-inflammatory activities.

Zingiber officinale, Zingiberaceae

Ginger is the common name for the monocotyledonous perennial plant Zingiber officinale. The term is also used to describe the edible rhizome part of the plant which is commonly used as a spice in cooking throughout the world. The ginger plant has a long history of cultivation known to originate in China and then spread to India, Southeast Asia, West Africa, and the Caribbean.

Ginger contains up to 3% of an essential oil that causes the fragrance of the spice. The main constituents are sesquiterpenoids with (−)-zingiberene as the main component. The pungent taste of ginger is due to nonvolatile phenylpropanoid-derived compounds, particularly gingerols and shogaols. The latter are formed from the former when ginger is dried or cooked. Zingerone is also produced from gingerols during this process, and it is less pungent and has a spicy-sweet aroma.

Ginger is also made into candy and used as a flavoring for cookies, crackers and cake, and is the main flavor in ginger ale—a sweet, carbonated, non-alcoholic beverage, as well as the similar, but somewhat spicier beverage ginger beer.

Ginger was classified as a stimulant and carminative, and used frequently for dyspepsia and colic. It was also frequently employed to disguise the taste of medicines. Ginger is on the FDA's ‘generally recognized as safe’ list, though it does interact with some medications, including warfarin. Ginger is contraindicated in people suffering from gallstones as the herb promotes the release of bile from the gallbladder. Ginger may also decrease joint pain from arthritis, though studies on this have been inconsistent, and may have blood thinning and cholesterol lowering properties that may make it useful for treating heart disease.

Ginger gives relief from muscular discomfort and pain. It inhibits prostaglandin and leukotriene biosynthesis and histamine release. Thus it acts as an anti-inflammatory as well as an antacid agent. It is a dual inhibitor of the lipoxigenase and cycloxigenase system.

The characteristic odor and flavor of ginger root is caused by a mixture of zingerone, shogaols and gingerols, volatile oils that compose about one to three percent of the weight of fresh ginger. In laboratory animals, the gingerols increase the motility of the gastrointestinal tract and have analgesic, sedative, antipyretic and antibacterial properties.

### Beta Glucan

Amylodextrins, baker's yeast, barley, beta-glucans, beta glycans, beta-glycans, grifolan (GRN), griton-d(r) (GD), d-fraction, lentinan, shiitake mushroom, maitake mushroom, PGG glucan, PGG-glucan, oat beta-glucan, oat fiber, oat fiber, oat gum, Plantago major L., Poria cocos sclerotium, Saccharomyces cerevisiae, schizophyllan (SPG), Sparassis crispa, SSG, yeast-derived beta glucan.

Beta-glucan is a soluble fiber derived from the cell walls of algae, bacteria, fungi, yeast, and plants. It is commonly used for its cholesterol-lowering effects. Beta-glucans have also been used to treat diabetes and for weight loss.

Concentrated yeast-derived beta-glucan is more easily incorporated into food products than grain beta-glucans, which are found in cereal grains like oat and barely. Yeast-derived beta-glucan is also more palatable than oat because it is not soluble in water and does not become viscous in water as beta-glucan from oats does. However, oat beta-glucan may have a higher therapeutic benefit potential.

The use of beta-glucan is a relatively new practice. Practitioners have used beta-glucan as an immunostimulant or as an adjunct cancer treatment. Beta-glucan is also used for its cholesterol-lowering effects and glycemic (blood sugar) control. In 1997, the U.S. Food and Drug Administration (FDA) passed a ruling that allowed oat bran to be registered as the first cholesterol-reducing food at an amount of 3 grams beta-glucan daily.

### Cod Liver Oil

Cod liver oil is a nutritional supplement that is derived from cod livers. It has high levels of the omega-3 fatty acids, EPA and DHA, and very high levels of vitamin A, and vitamin D. It is widely taken to ease the symptoms of arthritis. It was commonly given to children.

Cod liver oil is widely taken to ease the pain and joint stiffness associated with arthritis, but has also been clinically proven to have a positive effect on heart, bone, and brain, as well as helping to nourish skin, hair, and nails. Cod liver oil and fish oil are similar, but cod liver oil has much higher levels of vitamins A and D. Many adults don't meet the RDA for Vitamin D.

### Vitamin C

Vitamin C or L-ascorbate is an essential nutrient for higher primates, and a small number of other species. The presence of ascorbate is required for a range of essential metabolic reactions in all animals and plants. It is made internally by almost all organisms, humans being one notable exception. It is widely known as the vitamin whose deficiency causes scurvy in humans. It is also widely used as a food additive.

The pharmacophore of vitamin C is the ascorbate ion. In living organisms, ascorbate is an antioxidant, as it protects the body against oxidative stress, and is a cofactor in several vital enzymatic reactions.

In humans, vitamin C is a highly effective antioxidant, acting to lessen oxidative stress, a substrate for ascorbate peroxidase, as well as an enzyme cofactor for the biosynthesis of many important biochemicals. Vitamin C acts as an electron donor for eight different enzymes.

Since its discovery vitamin C has been considered by some enthusiastic proponents a “universal panacea”, although this led to suspicions by others that its effects were overvalued. Other proponents of high dose vitamin C consider that if it is given “in the right form, with the proper technique, in frequent enough doses, in high enough doses, along with certain additional agents and for a long enough period of time,” it can prevent and, in many cases, cure, a wide range of common and/or lethal diseases, notably the common cold and heart disease, although the NIH considers there to be fair scientific evidence against this use.

### Tocopherol—Vitamin E

Tocopherol, a class of chemical compounds of which many have vitamin E activity, describes a series of organic compounds consisting of various methylated phenols. The compound α-tocopherol, a common form of tocopherol added to food products, is denoted by the vitamin E number E307.

In foods, the most abundant sources of vitamin E are vegetable oils such as palm oil, sunflower, corn, soybean, and olive oil. Nuts, sunflower seeds, seabuckthorn berries, kiwi fruit, and wheat germ are also good sources. Other sources of vitamin E are whole grains, fish, peanut butter and green leafy vegetables.

There are three specific situations when a vitamin E deficiency is likely to occur. It is seen in persons who cannot absorb dietary fat, has been found in premature, very low birth weight infants (birth weights less than 1500 grams, or 3.5 pounds), and is seen in individuals with rare disorders of fat metabolism. A vitamin E deficiency is usually characterized by neurological problems due to poor nerve conduction.

Individuals who cannot absorb fat may require a vitamin E supplement because some dietary fat is needed for the absorption of vitamin E from the gastrointestinal tract. Anyone diagnosed with cystic fibrosis, individuals who have had part or all of their stomach removed, and individuals with malabsorptive problems such as Crohn's disease, liver disease or pancreatic insufficiency may not absorb fat and should discuss the need for supplemental vitamin E with their physician (3). People who cannot absorb fat often pass greasy stools or have chronic diarrhea.

Vitamin E is widely used in industry as an inexpensive preservative (namely for cosmetics and foods), scar reducing agent in cosmetics, preventive measure against coronary heart disease and blood clot formation, preventive measure against glaucoma, cataract and age-related macular degeneration in the presence of Vitamin C.

Vitamin E is an important regulator of prostaglandin E2 (PGE2), which plays a key role in inflammation and diseases associated with inflammation. Specifically, vitamin E inhibits cyclooxygenase-2 (COX-2) enzyme activity that promotes inflammatory response by catalyzing the synthesis of PGE2. Further, vitamin E enhances the T-cell function needed to inhibit the production of the pro-inflammatory Interleukin-1, which is responsible for inhibiting lacrimal aqueous secretion. Finally, vitamin stabilizes and prevents the oxidation of the omega-3 and omega-6 EFAs that are needed to generate anti-inflammatory PGE1.

### SUMMARY OF THE INVENTION

The compositions of the invention would be useful for, but not limited to, the treatment of inflammation-associated disorders, such as, as an analgesic in the treatment of pain and headaches, or as an antipyretic for the treatment of fever, arthritis, including but not limited to rheumatoid arthritis, spondyloathopathies, gouty arthritis, osteoarthritis, systemic lupus erythematosus, and juvenile arthritis, asthma, bronchitis, menstrual cramps, tendonitis, bursitis, and skin related conditions such as psoriasis, eczema, burns and dermatitis, and also to treat gastrointestinal conditions such as inflammatory bowel disease, Crohn's disease, gastritis, irritable bowel syndrome and ulcerative colitis and for the prevention or treatment of cancer such as colorectal cancer and other inflammatory condition in such diseases as vascular diseases, migraine headaches, periarteritis nodosa, thyroiditis, aplastic anemia, Hodgkin's disease, sclerodma, rheumatic fever, type I diabetes, myasthenia gravis, multiple sclerosis, sacoidosis, nephrotic syndrome, Behchet's syndrome, polymyositis, gingivitis, hypersensitivity, swelling occurring after injury, myocardial ischemia and the like.

The compositions of the present invention may also be useful in the treatment of ophthalmic diseases, such as retinopathies, conjunctivitis, uveitis, ocular photophobia, and of acute injury to the eye tissue. The combined component compositions would also be useful in the treatment of pulmonary inflammation, such as that associated with viral infections and cystic fibrosis. The combinations of components would also be useful for the treatment of certain nervous system disorders such as cortical dementias, including Alzheimer's disease. As inhibitors of COX-2 mediated biosynthesis of PGE2, these compositions may also be useful in the treatment of allergic rhinitis, respiratory distress syndrome, endotoxin shock syndrome, atherosclerosis, and central nervous system damage resulting from stroke, ischemia and trauma.

Compositions comprising herbal combinations that show synergistic anti-inflammatory effect have not been extensively described in either traditional or commercial medicine. Therefore, it is of interest and is useful to identify compositions that would synergistically enhance the anti-inflammatory effect of herbal combinations so that they could be used at sufficiently low doses without adverse side effects.

Thus, it would be useful to identify a natural formulation and/or combination of herbs that would specifically inhibit or prevent the synthesis of prostaglandins by COX-2. The composition of present invention represent methods useful for inflammation-induced pain-associated conditions and preserving the health of joint tissues, for treating arthritis or other inflammatory conditions, has not previously been discussed.

A unique feature of the present disclosure is the use of herbs and the constituents thereof that show synergistic anti-inflammatory effect in specific combination with the other components of the formulation.

Feature of the present disclosure is the use of baicalein, baicalin, beta-glucan, beta-sitosterol, cinnamaldehydes, curcuminoids, DHA, DHEA, EPA, gingerols, harpagoside, omega-3 fatty acids, omega-6 fatty acids, oroxylin A, resveratrol, shogaols, vitamin-C, vitamin-E, wogonin, and others listed in Table 1 in various combinations against inflammation-associated disorders. Particularly preferred compositions include those comprising the combination of 6-shogaol, curcumin and resveratrol (most preferably in a weight ratio of 33/33/33) and the combination of DHA, curcumin and 6-shogaol (most preferably in a weight ratio of 33/33/33).

TABLE 1
A list of active constituents found in the herbs.

| | |
|---|---|
| S. baicalensis: | baicalin, baicalein, wogonin, scutellarein, |
| | neobaicalein, skullcapflavone |
| C. longa: | curcumin, demothoxycurcumin, bis-demethoxycur- |
| | cumin, calebin-A, and other curcuminoids |
| Z. officinale: | gingerols, gingerone, shogaols |
| R. officinalis: | carnosol, carnosic acid, ferulic acid, |
| | catechin, epicatechin, ursolic acid, |
| | rosmarinic acid |
| S. officinalis: | 12-O-methyl carnosol, rosmanol, carnosol, |
| | epirosmanol, isorosmanol, galdosol, carnosic |
| | acid, miltirone, atuntzensin A, luteolin, 7-O- |
| | methyl luteolin, eupafolin, salvigenin, stig- |
| | masterol, lupeol, thujone, 7-methoxyrosmanol, |
| | oleanolic acid, royleanonic acid, geraniol |
| T. parthenium: | parthenolide |
| H. procumbens: | harpagoside, beta-sitosterol, harpagide, 8- |
| | coumaroylharpagide, verbascoside |
| C. verum: | O-methoxycinnamaldehyde, cinnamaldehyde, cinna- |
| | myl alcohol, coumarin, cinnamic acid, eugenol |

Another feature of the present disclosure is the use of dried powder of S. baicalensis, L. chinense, C. chinensis, C. longa, R. nigrum, A. membranaceus, A. macrocephala, R. officinalis, S. officinalis, Z. officinale, A. ovata, flax seed, T. parthenium, H. procumbens, C. sinensis, C. verum, T. vulgare, nuts, fish oil, edible mushrooms and other beta-glucan sources, and etc in various combinations against inflammation-associated disorders.

Another feature of the present disclosure is the use of extract of S. baicalensis, L. chinense, C. chinensis, C. longa, R. nigrum, A. membranaceus, A. macrocephala, R. officinalis, S. officinalis, Z. officinale, A. ovata, flax seed, T. parthenium, H. procumbens, C. sinensis, C. verum, T. vulgare, nuts, fish oil, edible mushrooms and other beta-glucan sources, and etc in various combinations against inflammation-associated disorders. Particularly preferred formulations include the combination of C. verum; C. longa and Z. officinale (most preferably in a weight ratio of 33/33/33) and the combination of S. baicalensis; C. longa and Z. officinale (most preferably in a weight ratio of 33/33/33).

Another feature of the present disclosure is the use of S. baicalensis, L. chinense, C. chinensis, C. longa, R. nigrum, A. membranaceus, A. macrocephala, R. officinalis, S. officinalis, Z. officinale, A. ovata, flax seed, T. parthenium, H. procumbens, C. sinensis, C. verum, T. vulgare, nuts, fish oil, edible mushrooms and other beta-glucan sources, and etc and baicalein, baicalin, beta-glucan, beta-sitosterol, cinnamaldehyde, curcuminoids, DHA, DHEA, EPA, gingerols, harpagoside, omega-3 fatty acids, omega-6 fatty acids, oroxylin A, resveratrol, shogaols, vitamin-C, vitamin-E, wogonin, camosol, camosolic acid, rosmarinic acid, parthenolide and others listed in Table 2 in various combinations against inflammation-associated disorders.

Another feature of the present disclosure suggests the use of Astragalus sp., Atractylodes sp., Camellia sp., Cinnamomum sp., Curcuma sp., Cuscuta sp., Harpagophytum sp., Lycium sp., Ribes sp., Rosmarinus sp., Salvia sp., Scutellaria sp., Tanacetum sp., Zingiber sp. may likely yield similar or comparable results disclosed herein.

Another feature of the present disclosure suggests the use of Astragalus sp., Atractylodes sp., Camellia sp., Cinnamomum sp., Curcuma sp., Cuscuta sp., Harpagophytum sp., Lycium sp., Ribes sp., Rosmarinus sp., Salvia sp., Scutellaria sp., Tanacetum sp., Zingiber sp. in the presence of constituents such as omega-3-fatty acids etc also presented in this closure may likely yield similar or comparable results disclosed herein.

In particular the invention provides, a formulation of an effective amount of baicalein, baicalin, beta-glucan, beta-sitosterol, cinnamaldeyde, curcuminoids, DHA, DHEA, EPA, gingerols, harpagoside, omega-3 fatty acids, omega-6 fatty acids, oroxylin A, resveratrol, shogaols, wogonin, camosol, camosolic acid, rosmarinic acid, parthenolide and etc in various combinations against inflammation-associated disorders; more specifically, a combination of cinnamaldehyde, curcuminoids, shogaols and/or gingerols, with or without omega-3 fatty acids (DHA, EPA and others, in particular, DHA); a combination of cinnamaldehydes, curcuminoids, shogaols and/or gingerols, harpagosides, with or without omega-3 fatty acids; a combination of cinnamaldehydes, curcuminoids, shogaols and/or gingerols, baicalein and/or baicalin, with or without omega-3 fatty acids; a combination of cinnamaldehydes, curcuminoids, shogaols and/or gingerols, with or without omega-3 fatty acids; a combination of cinnamaldehydes, curcuminoids, shogaols and/or gingerols, parthenolide, harpagoside with or without and omega-3 fatty acids; along with other constituents list above as additional components. Also provided are formulations of an effective amount of dried powder of S. baicalensis, L. chinense, C. chinensis, C. longa, R. nigrum, A. membranaceus, A. macrocephala, R. officinalis, S. officinalis, Z. officinale, A. ovata, flax seed, T. parthenium, H. procumbens, C. sinensis, C. verum, T. vulgare, nuts, fish oil, edible mushrooms and other beta-glucan sources, and etc in various combinations against inflammation-associated disorders; more specifically, a combination of C. longa, Z. officinale, S. baicalensis, and C. verum; a combination of C. longa, Z. officinale, T. parthenium, and C. verum; a combination of C. longa, Z. officinale, H procumgens, and C. verum; a combination of C. longa, Z. officinal, S officinalis or R. officinalis, and C. verum; along with other herbs listed herein in various amount, as minor constituents; More particularly preferred combinations are: C. longa, Z. officinale, S. baicalensis, C. verum with/without omega-3 fatty acids such as DHA or the natural sources that contain high quantities of omega-3 fatty acids such as flax seed oil, fish oil, and nuts; C. longa, Z. officinale, T. parthenium, C. verum with and without omega-3 fatty acids such as DHA, EPA or the natural sources that contain high quantities of omega-3 fatty acids such as flax seed oil, fish oil, and nuts; C. longa, Z. officinale, H. procumbens, C. verum with and without omega-3 fatty acids such as DHA, EPA or the natural sources that contain high quantities of omega-3 fatty acids such as flax seed oil, fish oil and nuts; C. longa, Z. officinale, S officinalis or R. officinalis, C. verum with and without omega-3 fatty acids such as DHA, EPA or the natural sources that contain high quantities of omega-3 fatty acids such as flax seed oil, fish oil, and nuts.

Another formulation is provided of an effective amount of extracts of S. baicalensis, L. chinense, C. chinensis, C. longa, R. nigrum, A. membranaceus, A. macrocephala, R. officinalis, S. officinalis, Z. officinale, A. ovata, flax seed, T. parthenium, H. procumbens, C. sinensis, C. verum, T. vulgare, nuts, fish oil, edible mushrooms and other beta-glucan sources, and etc in various combinations against inflammation-associated disorders; more specifically, a combination of C. longa, Z. officinale, S. baicalensis, and C. verum; a combination of C. longa, Z. officinale, T. parthenium, and C. verum; a combination of C. longa, Z. officinale, H procumbens, and C. verum; a combination of C. longa, Z. officnale, S officinalis or R. officinalis, and C. verum; along with other herbs listed herein in various amount, as minor constituents. More particularly preferred combinations are: C. longa, Z. officinale, S. baicalensis, C. verum with and without omega-3 fatty acids such as DHA, EPA or the natural sources that contain high quantities of omega-3 fatty acids such as flax seed oil, fish oil, and nuts; a combination of C. longa, Z. officinale, T. parthenium, C. verum and omega-3 fatty acids such as DHA, EPA or the natural sources that contain high quantities of omega-3 fatty acids such as flax seed oil, fish oil, and nuts; C. longa, Z. officinale, H. procumbens, C. verum with and without omega-3 fatty acids such as DHA, EPA or the natural sources that contain high quantities of omega-3 fatty acids such as flax seed oil, fish oil, and nuts; C. longa, Z. officnale, S officinalis or R. officinalis, C. verum with and without fatty acids such as DHA, EPA or the natural sources that contain high quantities of omega-3 fatty acids such as flax seed oil, fish oil, and nuts.

Preferred extracts for use with the invention are prepared according to the following method wherein Ground herbs (500 g) were slowly stirred in 90% aqueous methanol (1000 mL) overnight and filtered. The process was repeated thrice. Solvent was evaporated under vacuum and the residue was reconstituted with 90% aqueous methanol (200 mL), filtered, rinsed with 90% aqueous methanol (25 mL), solvent was evaporated under vacuum, chloroform (100 mL) was added and stirred, residue was filtered, and organic layer was washed with water (3×100 mL). Water layer was back extracted with chloroform (2×50 mL). Chloroform layers were combined dried (MgSO₄), filtered, and the solvent was evaporated under vacuum to give residue.

Extracts were also prepared using 90% aqueous isopropyl alcohol and 90% aqueous ethanol to compare the extraction efficacy. The amount of residue from chloroform extraction of herbs using 90% aqueous isopropyl alcohol was comparable to those of 90% aqueous ethanol or 90% aqueous methanol. All extractions were performed at room temperature and the vacuum evaporation was carried out at temperature below 50° C.

Still other formulations comprise an effective amount of dried powder of S. baicalensis, L. chinense, C. chinensis, C. longa, R. nigrum, A. membranaceus, A. macrocephala, R. officinalis, S. officinalis, Z. officinale, A. ovata, flax seed, T. parthenium, H. procumbens, C. sinensis, C. verum, T. vulgare, nuts, fish oil, edible mushrooms and other beta-glucan sources, and etc and baicalein, baicalin, beta-glucan, beta-sitosterol, curcuminoids, DHA, DHEA, EPA, gingerols, harpagoside, omega-3-acids, omega-6-acids, oroxylin A, resveratrol, shogaols, wogonin, carnosol, carnosolic acid, rosmarinic acid, parthenolide and etc in various combinations against inflammation-associated disorders; more specifically, a combination of C. longa, Z. officinale, S. baicalensis, and C. verum; a combination of C. longa, Z. officinale, T. parthenium, and C. verum; a combination of C. longa, Z. officinale, H. procumbens, and C. verum; a combination of C. longa, Z. officinale, S officinalis or R. officinalis, or R. officinalis and C. verum; along with other herbs listed herein in various amount, as minor constituents; more particularly preferred combinations are C. longa, Z. officinale, S. baicalensis, C. verum with and without omega-3 fatty acids such as DHA, EPA or the natural sources that contain high quantities of omega-3 fatty acids such as flax seed oil, fish oil, and nuts; C. longa, Z. officinale, T. parthenium, C. verum with and without omega-3 fatty acids such as DHA, EPA or the natural sources that contain high quantities of omega-3 fatty acids such as flax seed oil, fish oil, and nuts; C. longa, Z. officinale, H. procumbens, C. verum with and without omega-3 fatty acids such as DHA, EPA or the natural sources that contain high quantities of omega-3 fatty acids such as flax seed oil, fish oil, and nuts; C. longa, Z. officnale, S officinalis or R. officinalis, C. verum and omega-3 fatty acids such as DHA, EPA or the natural sources that contain high quantities of omega-3 fatty acids such as flax seed oil, fish oil, and nuts.

Other formulations include an effective amount of extracts of S. baicalensis, L. chinense, C. chinensis, C. longa, R. nigrum, A. membranaceus, A. macrocephala, R. officinalis, S. officinalis, Z. officinale, A. ovata, flax seed, T. parthenium, H. procumbens, C. sinensis, C. verum, T. vulgare, nuts, fish oil, edible mushrooms and other beta-glucan sources, and etc and baicalein, baicalin, beta-glucan, beta-sitosterol, curcuminoids, DHA, DHEA, EPA, gingerols, harpagoside, omega-3-acids, omega-6-acids, oroxylin A, resveratrol, shogaols, wogonin, camosol, carnosolic acid, rosmarinic acid, parthenolide and etc in various combinations against inflammation-associated disorders; more specifically, a combination of C. longa, Z. officinale, S. baicalensis, and C. verum; a combination of C. longa, Z. officinale, T. parthenium, and C. verum; a combination of C. longa, Z. officinale, H. procumbens, and C. verum; a combination of C. longa, Z. officnale, S officinalis or R. officinalis, and C. verum; along with other herbs listed herein in various amount, as minor constituents; more particularly preferred combinations are C. longa, Z. officinale, S. baicalensis, C. verum and omega-3 fatty acids such as DHA, EPA or the natural sources that contain high quantities of omega-3 fatty acids such as flax seed oil, fish oil, and nuts; C. longa, Z. officinale, T. parthenium, C. verum with and without omega-3 fatty acids such as DHA, EPA or the natural sources that contain high quantities of omega-3 fatty acids such as flax seed oil, fish oil, and nuts; C. longa, Z. officinale, H. procumbens, C. verum with and without omega-3 fatty acids such as DHA, EPA or the natural sources that contain high quantities of omega-3 fatty acids such as flax seed oil, fish oil, and nuts C. longa, Z. officnale, S officinalis or R. officinalis, C. verum and omega-3 fatty acids such as DHA, EPA or the natural sources that contain high quantities of omega-3 fatty acids such as flax seed oil, fish oil, and nuts.

Additional methods for preparing herbal extracts using various aqueous and organic solvents and/or aqueous organic solvents consisted of individual or combinations of two or more solvents selected from, but not limited to, water, alcohols such as methanol, ethanol, propyl alcohol and isopropyl alcohol, ketones that include acetone and methyl ethylketone, ethers such as diethyl ether, dimethyl ether and methylethyl ether, acetates that include ethyl acetate, acids such as acetic acid, anhydrides such as acetic anhydride, chlorinated solvents such as chloroform, dichloromethane and dichloroethane, and hexanes etc; passing through one or more columns of silica or polysaccharide based resins using appropriate eluents; and drying the said solution at various stages under vacuum or air to obtain processed extracts; combining the said extracts made using the solvents in various concentrations to treat inflammation-associated diseases.

Also provided are methods for treating at least one symptom of inflammatory condition listed herein by administering effective amount(s) of nutritional supplement(s) comprising herbal ingredients listed herein including a method for treating at least one symptom of inflammatory condition listed herein by administering effective amount(s) of herbal extract(s) in various combinations listed herein and methods for treating at least one symptom of inflammatory condition listed herein by administering effective amount(s) of powdered herbal ingredients in various combinations listed herein.

The invention also provides methods for treating at least one symptom of inflammatory condition listed herein by administering effective amount(s) of nutritional supplement constituent(s) in various combinations listed herein. Also provided is a method for treating at least one symptom of inflammatory condition listed herein by administering effective amount(s) of nutritional supplement constituent(s) in dried powder form and/or extract form and/or individual active constituent form in various combinations listed herein. Further provided is a method of treating inflammation-associated disorders, such as, as an analgesic in the treatment of pain and headaches, or as an antipyretic for the treatment of fever, arthritis, including but not limited to rheumatoid arthritis, spondyloathopathies, gouty arthritis, osteoarthritis, systemic lupus erythematosus, and juvenile arthritis, asthma, bronchitis, menstrual cramps, tendonitis, bursitis, and skin related conditions such as psoriasis, eczema, burns and dermatitis, and also to treat gastrointestinal conditions such as inflammatory bowel disease, Crohn's disease, gastritis, irritable bowel syndrome and ulcerative colitis and for the prevention or treatment of cancer such as colorectal cancer and other inflammatory condition in such diseases as vascular diseases, migraine headaches, periarteritis nodosa, thyroiditis, aplastic anemia, Hodgkin's disease, sclerodma, rheumatic fever, type I diabetes, myasthenia gravis, multiple sclerosis, sacoidosis, nephrotic syndrome, Behchet's syndrome, polymyositis, gingivitis, hypersensitivity, swelling occurring after injury, myocardial ischemia and the like.

The invention also provides a method of treating ophthalmic diseases, such as retinopathies, conjunctivitis, uveitis, ocular photophobia, and of acute injury to the eye tissue. The combined component compositions would also be useful in the treatment of pulmonary inflammation, such as that associated with viral infections and cystic fibrosis. The combinations of components would also be useful for the treatment of certain nervous system disorders such as cortical dementias, including Alzheimer's disease. As inhibitors of COX-2 mediated biosynthesis of PGE2, these compositions may also be useful in the treatment of allergic rhinitis, respiratory distress syndrome, endotoxin shock syndrome, atherosclerosis, and central nervous system damage resulting from stroke, ischemia and trauma.

### BRIEF DESCRIPTION OF THE FIGURES

FIG. 1 depicts the dose dependent response of inhibition of LPS-dependent nitrite (NO) release in the culture medium of activated macrophages by C. longa extract. Macrophages were incubated with LPS (10 μg/mL) and various concentrations (□g/mL) of C. longa extract for 18 hr. Experiments were repeated three times on different dates. Each data point represents the mean±SEM with P<0.05;

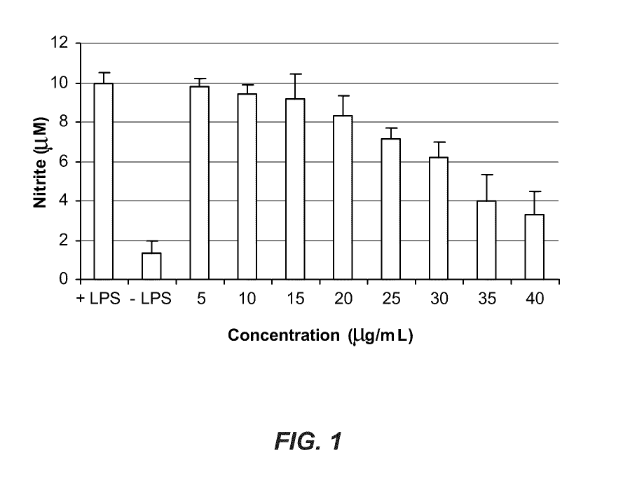

FIG. 2. depicts the dose dependent response of inhibition of LPS-dependent nitrite (NO) release in the culture medium of activated macrophages by S. officinalis extract. Macrophages were incubated with LPS (10 μg/mL) and various concentrations (D g/mL) of S. officinalis extract for 18 hr. Experiments were repeated three times on different dates. Each data point represents the mean±SEM with P<0.05.

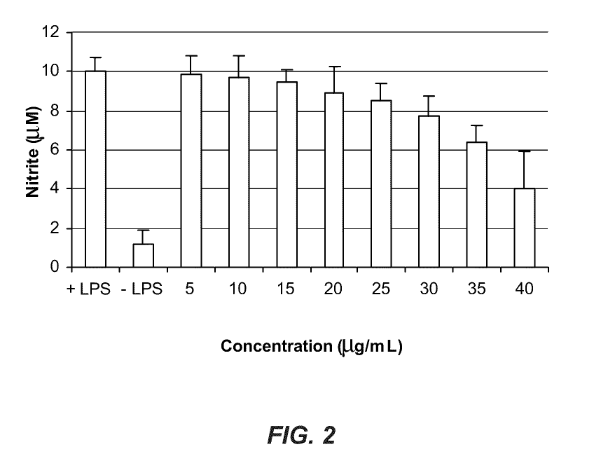

FIG. 3. depicts the dose dependent response of inhibition of LPS-dependent nitrite (NO) release in the culture medium of activated macrophages by Z. officinale extract. Macrophages were incubated with LPS (10 μg/mL) and various concentrations (μg/mL) of Z. officinale extract for 18 hr. Experiments were repeated three times on different dates. Each data point represents the mean±SEM with P<0.05;

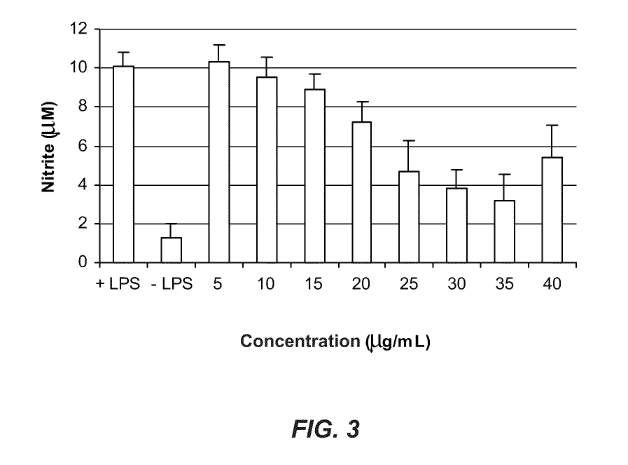

FIG. 4 depicts the dose dependent response of inhibition of LPS-dependent nitrite (NO) release in the culture medium of activated macrophages by DHA from flax seed oil. Macrophages were incubated with LPS (10 μg/mL) and various concentrations (μg/mL) of DHA for 18 hr. Experiments were repeated three times on different dates. Each data point represents the mean±SEM with P<0.05;

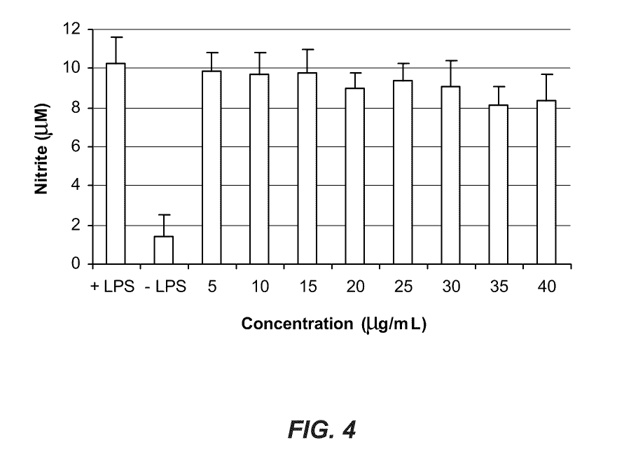

FIG. 5. depicts the dose dependent response of inhibition of LPS-dependent nitrite (NO) release in the culture medium of activated macrophages by S. baicalensis extract. Macrophages were incubated with LPS (10 μg/mL) and various concentrations (μg/mL) of S. baicalensis extract for 18 hr. Experiments were repeated three times on different dates. Each data point represents the mean±SEM with P<0.05;

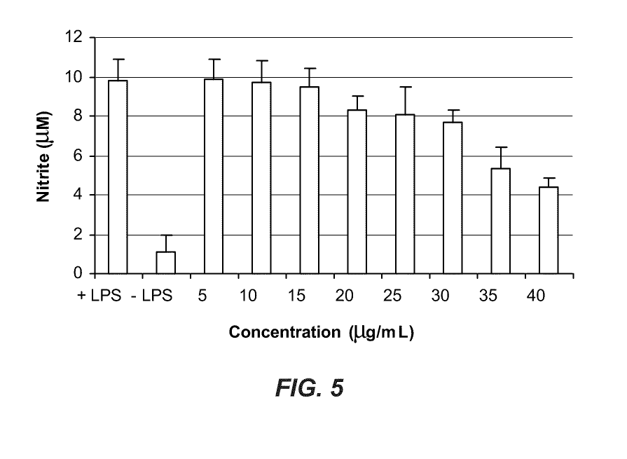

FIG. 6. depicts the dose dependent response of inhibition of LPS-dependent nitrite (NO) release in the culture medium of activated macrophages by H. procumbens extract. Macrophages were incubated with LPS (10 μg/mL) and various concentrations (μg/mL) of H. procumbens extract for 18 hr. Experiments were repeated three times on different dates. Each data point represents the mean±SEM with P<0.05

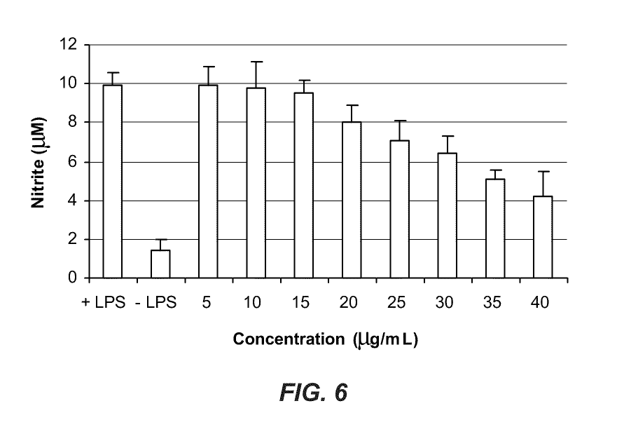

FIG. 7. depicts the dose dependent response of inhibition of LPS-dependent nitrite (NO) release in the culture medium of activated macrophages by C. verum extract. Macrophages were incubated with LPS (10 μg/mL) and various concentrations (μg/mL) of C. verum extract for 18 hr. Experiments were repeated three times on different dates. Each data point represents the mean±SEM with P<0.05.

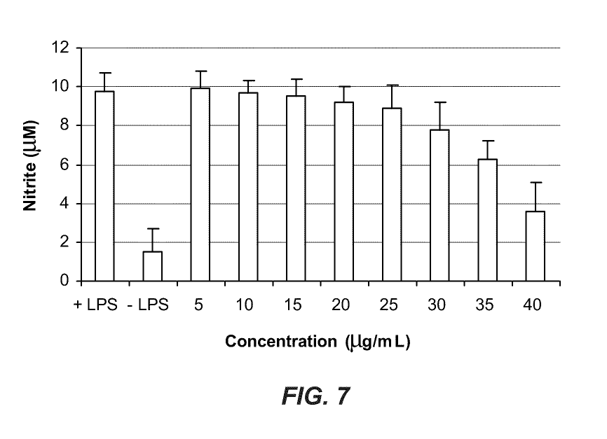

FIG. 8. depicts the dose dependent response of inhibition of LPS-dependent nitrite (NO) release in the culture medium of activated macrophages by C. longa, C. verum and (50/50) combined extracts. Macrophages were incubated with LPS (10 μg/mL) and various concentrations (μg/mL) of C. longa, C. verum and (50/50) combined extracts for 18 hr. Experiments were repeated three times on different dates. Each data point represents the mean±SEM with P<0.05.

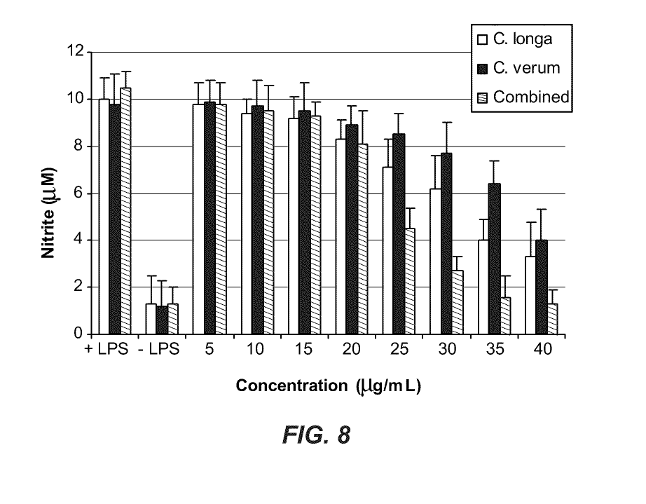

FIG. 9. depicts the dose dependent response of inhibition of LPS-dependent nitrite (NO) release in the culture medium of activated macrophages by C. longa extract, DHA from flax seed oil and (50/50) combined. Macrophages were incubated with LPS (10 μg/mL) and various concentrations (μg/mL) of C. longa extract, DHA from flax seed oil and (50/50) combined for 18 hr. Experiments were repeated three times on different dates. Each data point represents the mean±SEM with P<0.05.

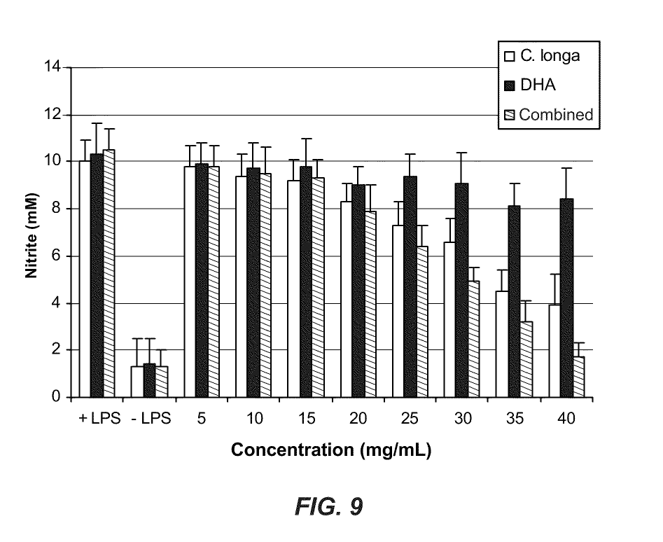

FIG. 10. depicts the dose dependent response of inhibition of LPS-dependent nitrite (NO) release in the culture medium of activated macrophages by C. longa, C. verum, Z. officinale and (33/33/33) combined extracts. Macrophages were incubated with LPS (10 μg/mL) and various concentrations (μg/mL) of C. longa, C. verum, Z. officinale and (33/33/33) combined extracts for 18 hr. Experiments were repeated three times on different dates. Each data point represents the mean±SEM with P<0.05.

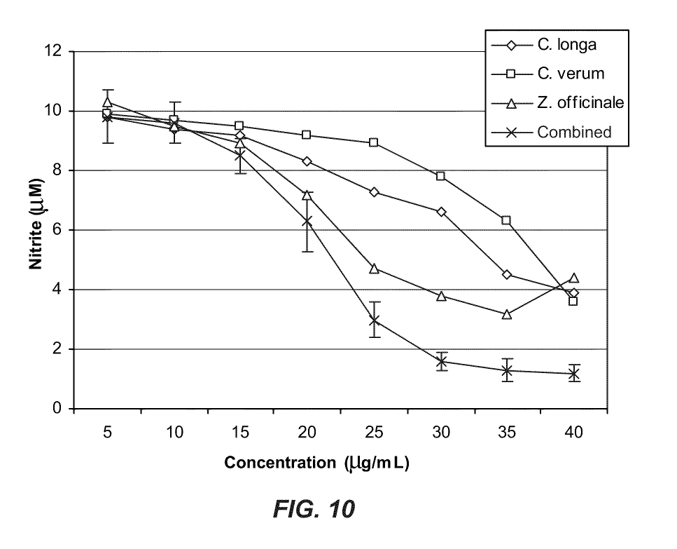

FIG. 11. depicts the dose dependent response of inhibition of LPS-dependent nitrite (NO) release in the culture medium of activated macrophages by 6-gingerol, curcumin, resveratrol and (33/33/33) combined compounds. Macrophages were incubated with LPS (10 μg/mL) and various concentrations (μg/mL) of 6-gingerol, curcumin, resveratrol and (33/33/33) combined compounds for 18 hr. Experiments were repeated three times on different dates. Each data point represents the mean±SEM with P<0.05.

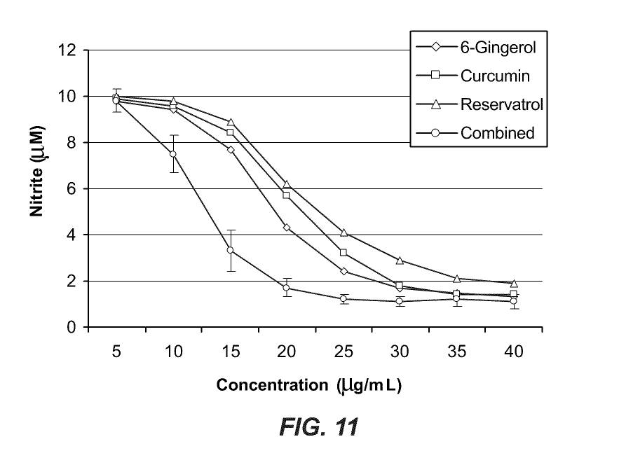

FIG. 12. depicts the dose dependent response of inhibition of LPS-dependent nitrite (NO) release in the culture medium of activated macrophages by C. longa, R. nigrum and (50/50 extracts. Macrophages were incubated with LPS (10 μg/mL) and various concentrations (μg/mL) of C. longa, R. nigrum and (50/50) combined extracts for 18 hr. Experiments were repeated three times on different dates. Each data point represents the mean±SEM with P<0.05.

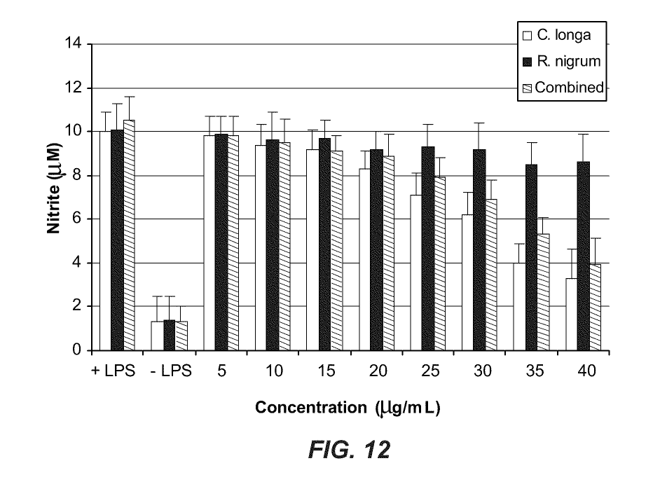

FIG. 13. depicts the dose dependent response of inhibition of LPS-dependent nitrite (NO) release in the culture medium of activated macrophages by vitamin-C, vitamin-E and (50/50) combined compounds. Macrophages were incubated with LPS (10 μg/mL) and various concentrations (μg/mL) of vitamin-C, vitamin-E and (50/50) combined compounds for 18 hr. Experiments were repeated three times on different dates. Each data point represents the mean±SEM with P<0.05.

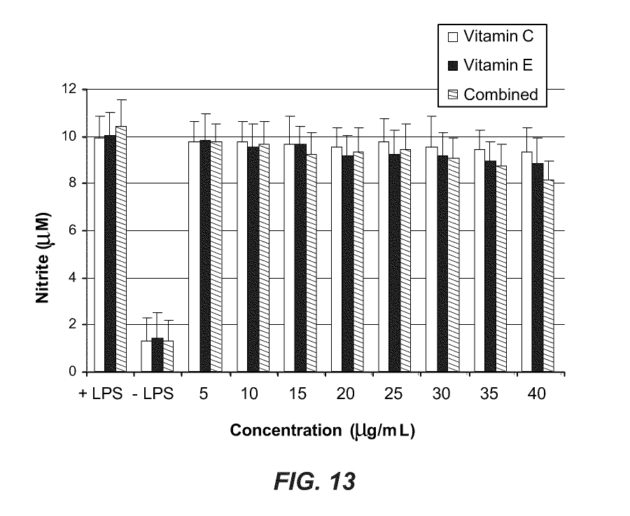

FIG. 14. depicts the dose dependent response of inhibition of LPS-dependent nitrite (NO) release in the culture medium of activated macrophages by L. chinense, C. verum and (50/50) combined extracts. Macrophages were incubated with LPS (10 μg/mL) and various concentrations (μg/mL) of L chinense, C. verum and (50/50) combined extracts for 18 hr. Experiments were repeated three times on different dates. Each data point represents the mean±SEM with P<0.05.

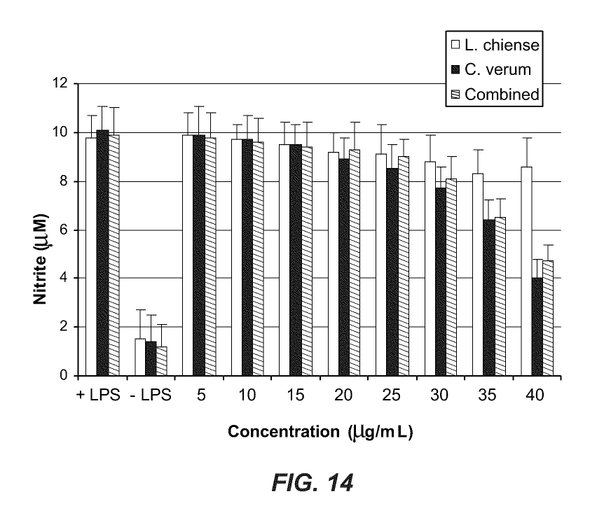

FIG. 15. depicts the dose dependent response of inhibition of LPS-dependent nitrite (NO) release in the culture medium of activated macrophages by A. membranaceus, C. chinensis and (50/50) combined extracts. Macrophages were incubated with LPS (10 μg/mL) and various concentrations (μg/mL) of A. membranaceus, C. chinensis and (50/50) combined extracts for 18 hr. Experiments were repeated three times on different dates. Each data point represents the mean±SEM with P<0.05.

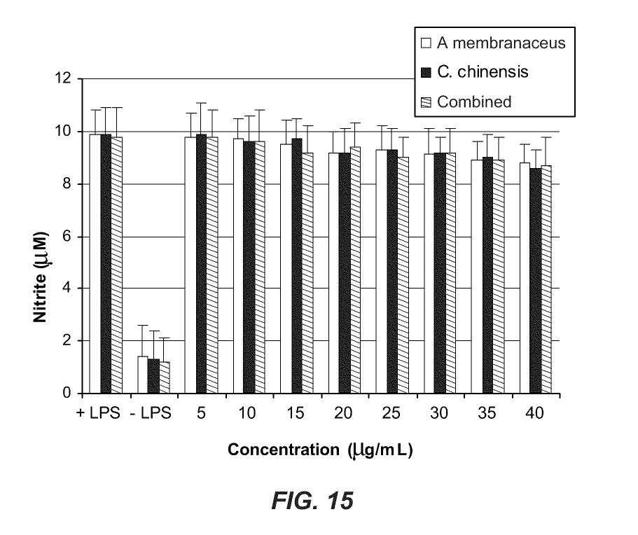

FIG. 16. depicts the dose dependent response of inhibition of LPS-dependent nitrite (NO) release in the culture medium of activated macrophages by A. macrocephala, S. baicalensis and (50/50) combined extracts. Macrophages were incubated with LPS (10 μg/mL) and various concentrations (μg/mL) of A. macrocephala, S. baicalensis and (50/50) combined extracts for 18 hr. Experiments were repeated three times on different dates. Each data point represents the mean±SEM with P<0.05.

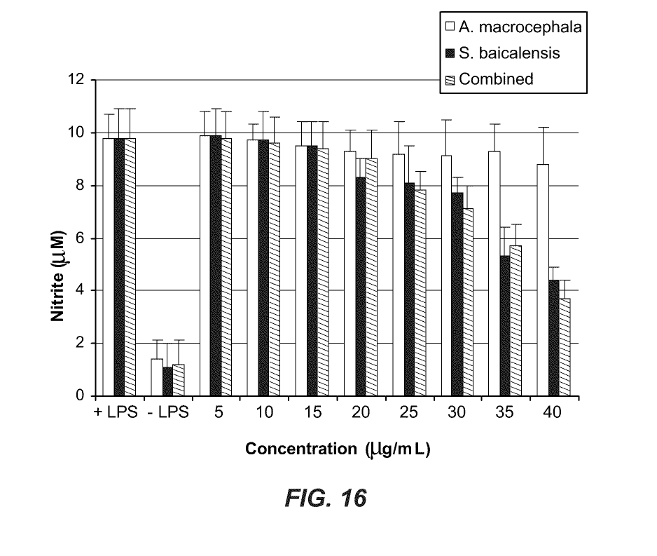

FIG. 17. depicts the dose dependent response of inhibition of LPS-dependent nitrite (NO) release in the culture medium of activated macrophages by T. parthenium, S. officinalis and (50/50) combined extracts. Macrophages were incubated with LPS (10 μg/mL) and various concentrations (μg/mL) of T. parthenium, S. officinalis and (50/50) combined extracts for 18 hr. Experiments were repeated three times on different dates. Each data point represents the mean±SEM with P<0.05.

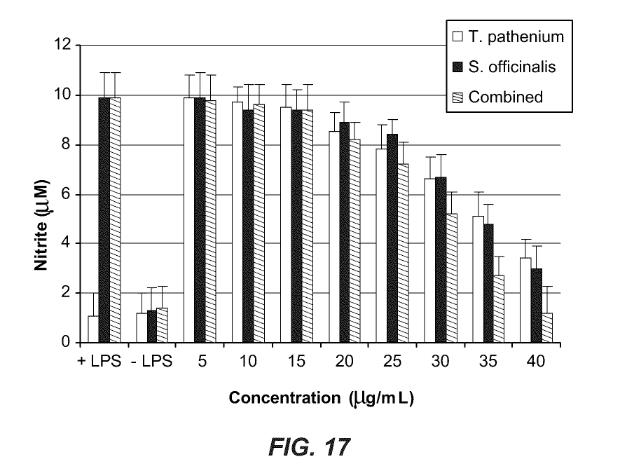

FIG. 18. depicts the dose dependent response of inhibition of LPS-dependent nitrite (NO) release in the culture medium of activated macrophages by S. officinalis, R. officinalis and (50/50) combined extracts. Macrophages were incubated with LPS (10 μg/mL) and various concentrations (μg/mL) of S. officinalis, R. officinalis and (50/50) combined extracts for 18 hr. Experiments were repeated three times on different dates. Each data point represents the mean±SEM with P<0.05.

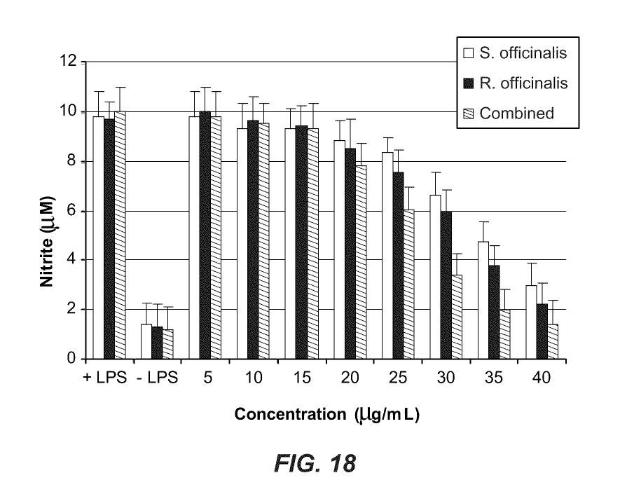

FIG. 19. depicts the dose dependent response of inhibition of LPS-dependent nitrite (NO) release in the culture medium of activated macrophages by C. sinensis, vitamin-E and (50/50) combined. Macrophages were incubated with LPS (10 μg/mL) and various concentrations (μg/mL) of C. sinensis, vitamin-E and (50/50) combined for 18 hr. Experiments were repeated three times on different dates. Each data point represents the mean±SEM with P<0.05.

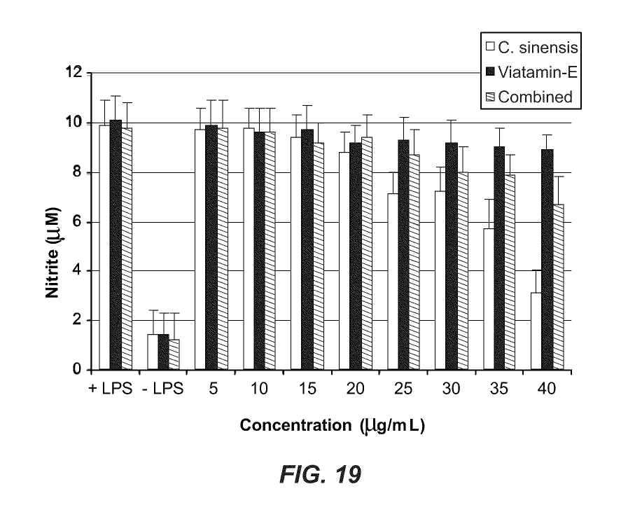

FIG. 20. depicts the dose dependent response of inhibition of LPS-dependent nitrite (NO) release in the culture medium of activated macrophages by C. longa, S. baicalensis, Z. officinale and (33/33/33) combined extracts. Macrophages were incubated with LPS (10 μg/mL) and various concentrations (μg/mL) of C. longa, S. baicalensis, Z. officinale and (33/33/33) combined extracts for 18 hr. Experiments were repeated three times on different dates. Each data point represents the mean±SEM with P<0.05.

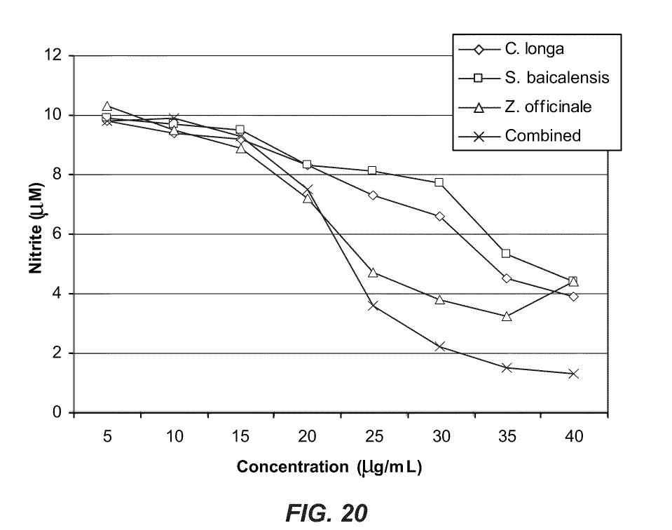

FIG. 21. depicts the dose dependent response of inhibition of LPS-dependent nitrite (NO) release in the culture medium of activated macrophages by C. longa, S. baicalensis, Z. officinale, C. verum and (25/25/25/25) combined extracts. Macrophages were incubated with LPS (10 μg/mL) and various concentrations (μg/mL) of C. longa, S. baicalensis, Z. officinale, C. verum and (25/25/25/25) combined extracts for 18 hr. Experiments were repeated three times on different dates. Each data point represents the mean±SEM with P<0.05.

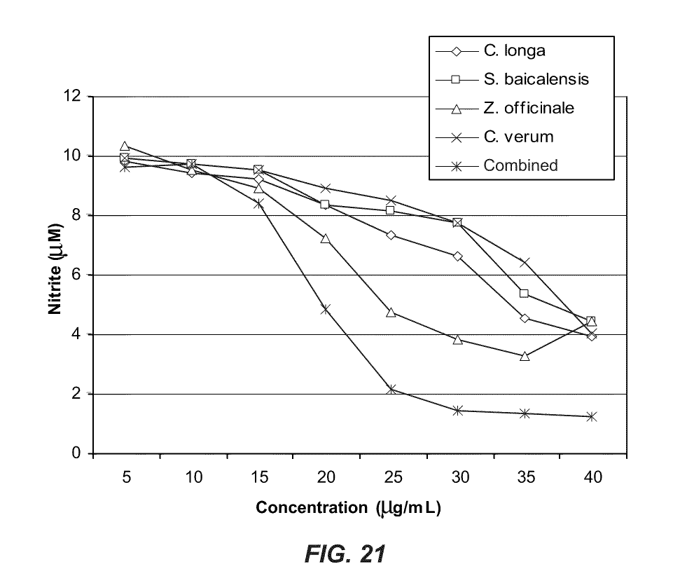

FIG. 22. depicts the dose dependent response of inhibition of LPS-dependent nitrite (NO) release in the culture medium of activated macrophages by C. longa, R. officinalis, Z. officinale, C. verum and (25/25/25/25) combined extracts. Macrophages were incubated with LPS (10 μg/mL) and various concentrations (μg/mL) of C. longa, R. officinalis, Z. officinale, C. verum and (25/25/25/25) combined extracts for 18 hr. Experiments were repeated three times on different dates. Each data point represents the mean±SEM with P<0.05.

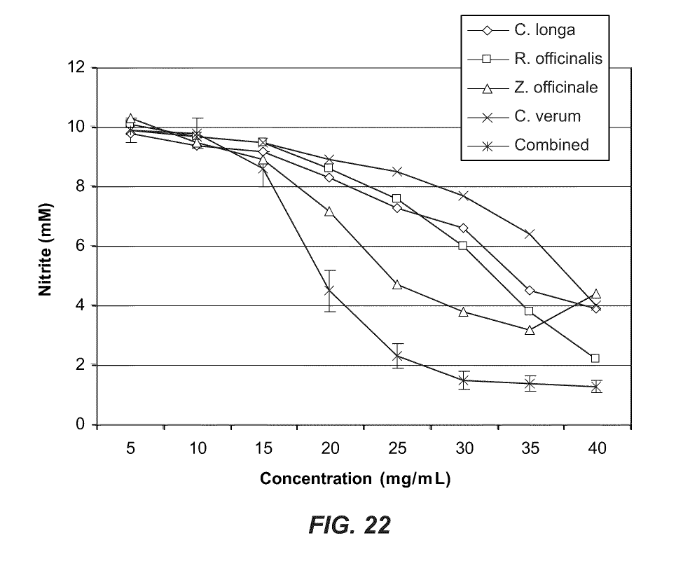

FIG. 23. depicts the dose dependent response of inhibition of LPS-dependent nitrite (NO) release in the culture medium of activated macrophages by C. verum, Z. officinale, T. parthenium, S. officinalis and (25/25/25/25) combined extracts. Macrophages were incubated with LPS (10 μg/mL) and various concentrations (μg/mL) of C. longa, Z. officinale, T. parthenium, S. officinalis and (25/25/25/25) combined extracts for 18 hr. Experiments were repeated three times on different dates. Each data point represents the mean±SEM with P<0.05.

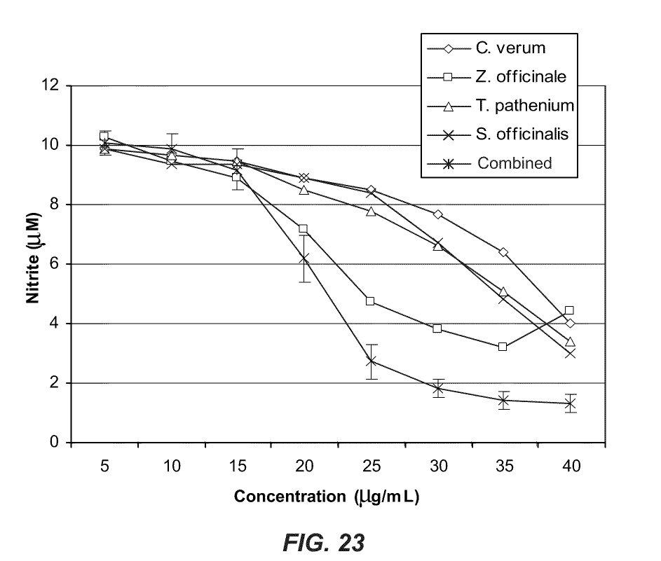

FIG. 24. depicts the dose dependent response of inhibition of LPS-dependent nitrite (NO) release in the culture medium of activated macrophages by C. longa, DHA, Z. officinale and (33/33/33) combined. Macrophages were incubated with LPS (10 μg/mL) and various concentrations (μg/mL) of C. longa, DHA, Z. officinale and (33/33/33) combined for 18 hr. Experiments were repeated three times on different dates. Each data point represents the mean±SEM with P<0.05.

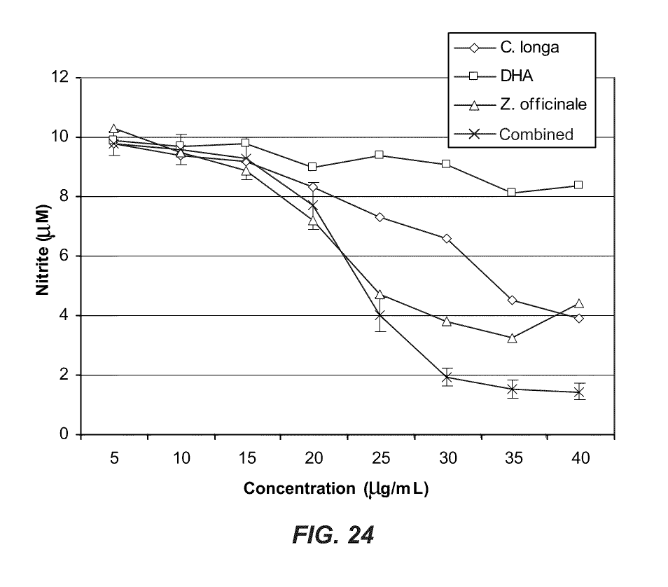

FIG. 25. depicts the dose dependent response of inhibition of LPS-dependent nitrite (NO) release in the culture medium of activated macrophages by C. longa, DHA, Z. officinale, C. verum and (25/25/25/25) combined. Macrophages were incubated with LPS (10 μg/mL) and various concentrations (μg/mL) of C. longa, DHA, Z. officinale, C. verum and (25/25/25/25) combined for 18 hr. Experiments were repeated three times on different dates. Each data point represents the mean±SEM with P<0.05.

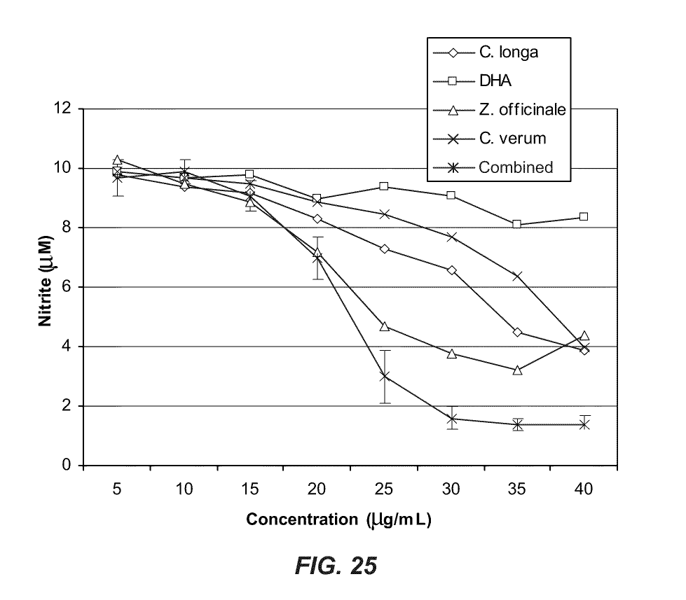

FIG. 26. depicts the dose dependent response of inhibition of LPS-dependent nitrite (NO) release in the culture medium of activated macrophages by 6-gingerol, curcumin, DHA and (33/33/33) combined compounds. Macrophages were incubated with LPS (10 μg/mL) and various concentrations (μg/mL) of 6-gingerol, curcumin, DHA and (33/33/33) combined compounds for 18 hr. Experiments were repeated three times on different dates. Each data point represents the mean±SEM with P<0.05.

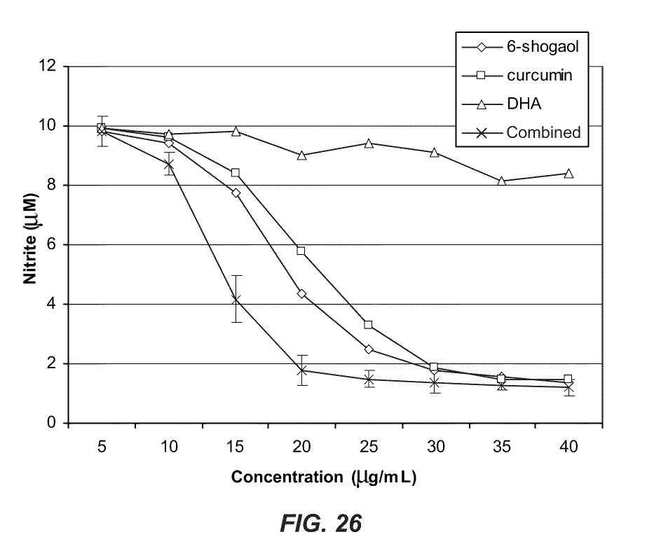

### DETAILED DESCRIPTION OF THE INVENTION

The present invention provides synergistic combinations of compounds having anti-inflammatory activity and methods for the therapeutic use of those combinations.

### Synergistic Effect

The term “synergy” refers to the phenomenon in which the combination of two or more discrete agents acting together can cause a greater response than simply the sum of the individual effects of each agent if they were used separately. It is not uncommon to find the presence of two or more chemical constituents or herbs on an organism to be greater than the effect of each chemical constituent or herb individually, or the sum of the individual effects. This enhancement of effect by the presence of two or more constituents is called a synergistic effect or synergy.
FIC_{A}=Nitrite concentration in combination/Nitrite concentration of sample A under investigation
FIC_{B}=Nitrite concentration in combination/Nitrite concentration of sample B under investigation
FIC_{C}=Nitrite concentration in combination/Nitrite concentration of sample C under investigation

and so on . . .

FICI=FIC_{A}+FIC_{B}+FIC_{C}+ . . .

mean FICI=FICI/number of samples used for FICI

Synergy=mean FICI≦0.5

No synergy=mean FICI>0.5

Antagonism=mean FICI>1

### Assay

### Materials and Methods

LPS (Escherichia coli 0127: E8) and chemicals were purchased from Sigma Chemical (St Louis, Mo.). Solvents were purchased from Fisher Scientific (Itasca, Ill.) and were used with further purification. Botanicals were purchased from Starwest Botanicals, Inc. (Rancho Cordova, Calif.).

### Preparation of Herbal Extracts

Ground herbs (500 g) were titrated in 90% aqueous methanol overnight (3×1000 mL). Solvent was evaporated under vacuum and the residue was reconstituted with 90% aqueous methanol (200 mL), filtered, rinsed with 90% aqueous methanol (25 mL), solvent was evaporated under vacuum, chloroform (100 mL) was added, residue was filtered, and organic layer was washed with water (3×100 mL). Water layer was back extracted with chloroform (2×50 mL). Chloroform layers were combined dried (MgSO₄), filtered, and the solvent was evaporated under vacuum to give residue.

The amount of residue from chloroform extraction of tritrated herbs using 90% aqueous isopropyl alcohol was comparable to those of 90% aqueous ethanol or 90% aqueous methanol. All extractions were performed at room temperature and the vacuum evaporation was carried out at temperature below 50° C.

### Cells

Murine macrophage RAW264.7 cell line was obtained from American Type Culture Collection (Rockville, Md.). RAW264.7 cell was maintained in Dulbecco's modified Eagle's medium (DMEM) containing L-glutamine, glucose and sodium bicarbonate, and McCoy 5A medium, respectively, supplemented with 10% fetal bovine serum, 100 U/ml penicillin and 0.1 mg/ml streptomycin, at 37° C. in 95% humidity and 5% CO₂. All other chemicals were purchased from Sigma Chemical (St Louis, Mo.).

Cell viability assay

RAW 264.7 cells were plated at a density of 10⁴ cells/well into 96-well plates. After overnight growth, cells were treated with a different concentration of flavonoids for 24 hr. At the end of treatment, 20 μL of combined solution of the tetrazolium compound MTT and an electron coupling reagent, phenazine methosulfate, were added to each well. After incubation for 1 hr at 37° in a humidified 5% CO₂ atmosphere, absorbance at 600 nm was recorded using an ELISA plate reader.

### Measurement of PGE2 Production

RAW 264.7 cells were plated at 1×10⁵ cells/well in a 12-well plate and incubated for 18 hr with testing samples in various concentration in the presence of LPS (500 ng/mL). One hundred microliters of supernatant of culture medium was collected for the determination of PGE₂ concentration by ELISA (Cayman Enzyme Immunoassay kit).

### Nitrite Assay

The nitrite concentration in the medium was measured as an indicator of NO production, a common phenomenon of the inflammation process, according to the Griess reaction. RAW 264.7 cells were plated at 1×10⁵ cells/well in a 12-well plate and incubated for 18 hr with testing samples in various concentrations in the presence of LPS (10 μg/mL). One hundred microliters of supernatant was mixed with the same volume of Griess reagent (1% sulfanilamide in 5% phosphoric acid and 0.1% naphthylethylenediamine dihydrocholide in water); absorbance of the mixture at 550 nm was determined with an ELISA plate reader (Dynatech MR-7000; Dynatech Laboratories Inc.).

### Statistics

The values are expressed as means±SEM. The significance of difference from the respective controls for each experimental test condition was assayed by using Student's t-test for each paired experiment. P values <0.05 were regarded as indicating significant differences.

### Discussion of Data

Various combinations of herbal extracts were investigated for anti-inflammatory effect using nitrite assay. For the study, RAW 264.7 cells were treated with LPS (10 μg/mL) to induce nitrite production, various concentrations and combinations thereof were added, and the nitrite concentration in the medium was measured as an indicator of the inhibition of NO production, a common phenomenon of the inflammation process.

As shown in Figures and Table 2, only selected extracts and the combinations showed inhibitory effect on NO production. Moreover, selected few combinations, in particular, various combinations of C. verum, C. longa, Z. officinale, S. baicalensis, T parthenium, R. officinalis, S. officinalis, even at combination of four different herbal extracts, showed remarkable synergistic effects against LPS induced NO production. Synergistic effects shown by DHA were particularly notable. Synergistic effects also shown by the combinations of curcumin, shogaol, DHA, and resveratrol were of particular interest for a pharmaceutical composition aspect. Vitamin C and vitamin E surprisingly did not show any noticeable anti-inflammatory property, either singly or in combination.

TABLE 2
Various combinations of herbal extracts and compounds show
synergistic inhibitory effect against LPS-induced nitrite
(NO) generation on murine macrophage RAW264.7 cells.

| | mean FICI value | | syn- |
|---|---|---|---|
| Herbal combinations (ratio) | (conc. μg/mL) | | ergy |
| C. verum/C. longa (50/50) | 0.40 (30) | | + |
| DHA/C. longa (50/50) | 0.32 (40) | | + |
| C. verum/C. longa/Z. officinale (33/33/33) | 0.29 (30) | | + |
| C. longa/R. nigrum (50/50) | 0.82 (40) | | none |
| Vitamin C/Vitamin E (50/50) | 0.90 (40) | | none |
| L. chinense/C. verum (50/50) | 0.87 (40) | | none |
| A. membranaceus/C. chinensis (50/50) | 1.00 (40) | | none |
| A. macrocephala/S. baicalensis (50/50) | 0.63 (40) | | none |
| T. parthenium/S. officinalis (50/50) | 0.38 (40) | | + |
| S. officinalis/R. officinalis (50/50) | 0.56 (40) | | none |
| C. sinensis/vitamin E (50/50) | 1.46 (40) | | none |
| S. baicalensis/C. longa/Z. officinale | 0.31 (40) | 0.40 (30) | + |
| (33/33/33) | | | |
| C. verum/C. longa/DHA/Z. officinale | 0.33 (30) | 0.28 (40) | + |
| (25/25/25/25) | | | |
| Z. officinale/DHA/C. longa (33/33/33) | 0.26 (30) | | + |
| 6-shogaol/curcumin/resveratrol (33/33/33) | 0.32 (20) | | + |
| DHA/curcumin/6-shogaol (33/33/33) | 0.29 (20) | | + |
| Z. officinale/C. longa/S. baicalensis/ | 0.24 (30) | 0.29 (40) | + |
| C. verum (25/25/25/25) | | | |
| R. officinalis/C. longa/Z. officinale/ | 0.27 (30) | 0.39 (40) | + |
| C. verum (25/25/25/25) | | | |
| C. verum/Z. officinale/T. parthenium/ | 0.31 (30) | 0.36 (40) | + |
| S. officinale (25/25/25/25) | | | |

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

What is claimed is:

1. A formulation comprising two or more members selected from the group consisting of baicalein, baicalin, beta-glucan, beta-sitosterol, cinnamaldeyde, curcuminoids, DHA, DHEA, EPA, gingerols, harpagoside, omega-3 fatty acids, omega-6 fatty acids, oroxylin A, resveratrol, shogaols, wogonin, carnosol, camosolic acid, rosmarinic acid, parthenolide which is effective against inflammation-associated disorders.

2. The formulation of claim 1 comprising the combination of 6-shogaol, curcumin and resveratrol.

3. The formulation of claim 1 comprising the combination of DHA, curcumin and 6-shogaol.

4. A formulation comprising two or more members selected from the group consisting of dried powder of S. baicalensis, L. chinense, C. chinensis, C. longa, R. nigrum, A. membranaceus, A. macrocephala, R. officinalis, S. officinalis, Z. officinale, A. ovata, flax seed, T. parthenium, H. procumbens, C. sinensis, C. verum, T. vulgare, nuts, fish oil, edible mushrooms and other beta-glucan sources which is effective against inflammation-associated disorders.

5. The formulation of claim 4 comprising the combination of C. verum, C. longa and Z. officinale.

6. The formulation of claim 4 comprising the combination of S. baicalensis, C. longa and Z. officinale.

7. A formulation comprising two or more members selected from the group consisting of extracts of S. baicalensis, L. chinense, C. chinensis, C. longa, R. nigrum, A. membranaceus, A. macrocephala, R. officinalis, S. officinalis, Z. officinale, A. ovata, flax seed, T. parthenium, H. procumbens, C. sinensis, C. verum, T. vulgare, nuts, fish oil, edible mushrooms and other beta-glucan sources, which is effective against inflammation-associated disorders.

8. The formulation of claim 7 comprising the combination of C. verum; C. longa and Z. officinale.

9. The formulation of claim 7 comprising the combination of S. baicalensis; C. longa and Z. officinale.

10. A formulation comprising two or more members selected from the group consisting of dried powder of S. baicalensis, L. chinense, C. chinensis, C. longa, R. nigrum, A. membranaceus, A. macrocephala, R. officinalis, S. officinalis, Z. officinale, A. ovata, flax seed, T. parthenium, H. procumbens, C. sinensis, C. verum, T. vulgare, nuts, fish oil, edible mushrooms and other beta-glucan sources, and two or more members selected from the group consisting of and baicalein, baicalin, beta-glucan, beta-sitosterol, curcuminoids, DHA, DHEA, EPA, gingerols, harpagoside, omega-3-acids, omega-6-acids, oroxylin A, resveratrol, shogaols, wogonin, carnosol, camosolic acid, rosmarinic acid, parthenolide which is effective against inflammation-associated disorders.

11. The formulation of claim 10 comprising the combination of C. verum; C. longa and Z. officinale.

12. The formulation of claim 10 comprising the combination of S. baicalensis; C. longa and Z. officinale.

13. A formulation of comprising two or more members selected from the group consisting of an effective amount of extracts of S. baicalensis, L. chinense, C. chinensis, C. longa, R. nigrum, A. membranaceus, A. macrocephala, R. officinalis, S. officinalis, Z. officinale, A. ovata, flax seed, T. parthenium, H. procumbens, C. sinensis, C. verum, T. vulgare, nuts, fish oil, edible mushrooms and other beta-glucan sources, and comprising two or more members selected from the group consisting of baicalein, baicalin, beta-glucan, beta-sitosterol, curcuminoids, DHA, DHEA, EPA, gingerols, harpagoside, omega-3-acids, omega-6-acids, oroxylin A, resveratrol, shogaols, wogonin, carnosol, carnosolic acid, rosmarinic acid, parthenolide which is effective against inflammation-associated disorders.

14. The formulation of claim 13 comprising the combination of C. verum; C. longa and Z. officinale.

15. The formulation of claim 13 comprising the combination of S. baicalensis; C. longa and Z. officinale.

16. A method of preparing herbal extracts using various aqueous and organic solvents and/or aqueous organic solvents consisted of individual or combinations of two or more solvents selected from, but not limited to, water, alcohols such as methanol, ethanol, propyl alcohol and isopropyl alcohol, ketones that include acetone and methyl ethylketone, ethers such as diethyl ether, dimethyl ether and methylethyl ether, acetates that include ethyl acetate, acids such as acetic acid, anhydrides such as acetic anhydride, chlorinated solvents such as chloroform, dichloromethane and dichloroethane, and hexanes etc; passing through one or more columns of silica or polysaccharide based resins using appropriate eluents; and drying the said solution at various stages under vacuum or air to obtain processed extracts; combining the said extracts made using the solvents in various concentrations to treat inflammation-associated diseases.

17. A method for treating at least one symptom of inflammatory condition by administering an effective amount of a composition according to one of claims 1, 4, 7, 10 and 13.

18. The method of claim 7 wherein the inflammation-associated disorder is selected from the group consisting of pain and headaches, fever, arthritis, including but not limited to rheumatoid arthritis, spondyloathopathies, gouty arthritis, osteoarthritis, systemic lupus erythematosus, and juvenile arthritis, asthma, bronchitis, menstrual cramps, tendonitis, bursitis, and skin related conditions such as psoriasis, eczema, burns and dermatitis, gastrointestinal conditions such as inflammatory bowel disease, Crohn's disease, gastritis, irritable bowel syndrome and ulcerative colitis, cancer such as colorectal cancer and other inflammatory condition in such diseases as vascular diseases, migraine headaches, periarteritis nodosa, thyroiditis, aplastic anemia, Hodgkin's disease, sclerodma, rheumatic fever, type I diabetes, myasthenia gravis, multiple sclerosis, sacoidosis, nephrotic syndrome, Behchet's syndrome, polymyositis, gingivitis, hypersensitivity, swelling occurring after injury, myocardial ischemia and pulmonary inflammation, such as that associated with viral infections and cystic fibrosis.

19. A method of treating ophthalmic diseases, such as retinopathies, conjunctivitis, uveitis, ocular photophobia, and of acute injury to the eye tissue by administering an effective amount of a composition according to one of claims 1, 4, 7, 10 and 13.

20. A method for the treatment of certain nervous system disorders such as cortical dementias, including Alzheimer's disease by administering an effective amount of a composition according to one of claims 1, 4, 7, 10 and 13.

21. A method for the treatment of allergic rhinitis, respiratory distress syndrome, endotoxin shock syndrome, atherosclerosis, and central nervous system damage resulting from stroke, ischemia and trauma by administering an effective amount of a composition according to one of claims 1, 4, 7, 10 and 13.
